(19) United States
(12) Patent Application Publication
(10) Pub. No.: US 2020/0069458 A1
(43) Pub. Date: Mar. 5, 2020

(54) COMPOSITIONS, TREATMENT SYSTEMS, AND METHODS FOR FRACTIONALLY FREEZING TISSUE

(71) Applicant: Zeltiq Aesthetics, Inc., Pleasanton, CA (US)

(72) Inventor: Linda Pham, Kensington, CA (US)

(21) Appl. No.: 16/557,814

(22) Filed: Aug. 30, 2019

Related U.S. Application Data
(60) Provisional application No. 62/725,782, filed on Aug. 31, 2018.

(51) Int. Cl. A61F 7/00; A61M 35/00

(52) U.S. Cl. CPC A61F 7/0085; A61F 7/007; A61M 35/00; A61F 2007/0052; A61M 2205/366; A61F 2007/0075; A61M 2205/3606; A61M 2205/3673; A61F 2007/0063

(57) ABSTRACT

Compositions and formulations having one or more anti-freeze proteins for use with devices and systems that enable tissue cooling, such as cryotherapy applications, for alteration and reduction of adipose tissue are described. Embodiments of the technology are further directed to methods, compositions having one or more anti-freeze proteins and devices that protect non-targeted cells from freeze damage during dermatological and related aesthetic procedures requiring sustained exposure to cold temperatures. Further embodiments of the technology include systems for enhancing sustained and/or replenishing release of a freezing point depressant having one or more anti-freeze proteins to a treatment site prior to and during cooling applications.

### CROSS-REFERENCE TO RELATED APPLICATION

The present application claims the benefit of the earlier filing date of U.S. Provisional Patent Application No. 62/725,782, filed March Aug. 31, 2018, which is incorporated herein by reference in its entirety.

### INCORPORATION BY REFERENCE OF COMMONLY OWNED APPLICATIONS AND PATENTS

U.S. Pat. No. 7,854,754 entitled “COOLING DEVICE FOR REMOVING HEAT FROM SUBCUTANEOUS LIPID-RICH CELLS”;

U.S. Pat. No. 8,337,539 entitled “COOLING DEVICE FOR REMOVING HEAT FROM SUBCUTANEOUS LIPID-RICH CELLS”;

U.S. Patent Publication No. 2013/0158636 entitled “COOLING DEVICE FOR REMOVING HEAT FROM SUBCUTANEOUS LIPID-RICH CELLS”;

U.S. Pat. No. 8,192,474 entitled “TISSUE TREATMENT METHODS”;

U.S. Patent Publication No. 2013/0066309 entitled “TISSUE TREATMENT METHODS”;

U.S. Patent Publication No. 2015/0328077 entitled “TISSUE TREATMENT METHODS”;

U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;

U.S. Pat. No. 9,375,345 entitled “COOLING DEVICE HAVING A PLURALITY OF CONTROLLABLE COOLING ELEMENTS TO PROVIDE A PREDETERMINED COOLING PROFILE”;

U.S. Publication No. 2015/0342780 entitled “COOLING DEVICE HAVING A PLURALITY OF CONTROLLABLE COOLING ELEMENTS TO PROVIDE A PREDETERMINED COOLING PROFILE”;

U.S. Patent Publication No. 2008/0077201 entitled “COOLING DEVICES WITH FLEXIBLE SENSORS”;

U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;

U.S. patent application Ser. No. 15/833,329 entitled “COMPOSITIONS, TREATMENT SYSTEMS AND METHODS FOR IMPROVED COOLING OF LIPID-RICH TISSUE;”

U.S. Patent Publication No. 2014/0005760 entitled “CRYOPROTECTANT FOR USE WITH A TREATMENT DEVICE FOR IMPROVED COOLING OF SUBCUTANEOUS LIPID-RICH CELLS”;

U.S. Patent Publication No. 2007/0270925 entitled “METHOD AND APPARATUS FOR NON-INVASIVELY REMOVING HEAT FROM SUBCUTANEOUS LIPID RICH CELLS INCLUDING A COOLANT HAVING A PHASE TRANSITION TEMPERATURE”;

U.S. Patent Publication No. 2009/0118722 entitled “METHOD AND APPARATUS FOR COOLING SUBCUTANEOUS LIPID-RICH CELLS OR TISSUE”;

U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Patent Publication No. 2013/0079684 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 8,285,390 entitled “MONITORING THE COOLING OF SUBCUTANEOUS LIPID-RICH CELLS, SUCH AS THE COOLING OF ADIPOSE TISSUE”;

U.S. Pat. No. 9,408,745 entitled “MONITORING THE COOLING OF SUBCUTANEOUS LIPID-RICH CELLS, SUCH AS THE COOLING OF ADIPOSE TISSUE”;

U.S. Pat. No. 9,408,745 entitled “MONITORING THE COOLING OF SUBCUTANEOUS LIPID-RICH CELLS, SUCH AS THE COOLING OF ADIPOSE TISSUE”;

U.S. Pat. No. 8,523,927 entitled “SYSTEM FOR TREATING LIPID-RICH REGIONS”;

U.S. Pat. No. 9,655,770 entitled “SYSTEM FOR TREATING LIPID-RICH REGIONS”;

U.S. Patent Publication No. 2009/0018624 entitled “LIMITING USE OF DISPOSABLE SYSTEM PATIENT PROTECTION DEVICES”;

U.S. Patent Publication No. 2009/0018625 entitled “MANAGING SYSTEM TEMPERATURE TO REMOVE HEAT FROM LIPID-RICH REGIONS”;

U.S. Patent Publication No. 2009/0018626 entitled “USER INTERFACES FOR A SYSTEM THAT REMOVES HEAT FROM LIPID-RICH REGIONS”;

U.S. Patent Publication No. 2009/0018627 entitled “SECURE SYSTEM FOR REMOVING HEAT FROM LIPID-RICH REGIONS”;

U.S. Pat. No. 8,275,442 entitled “TREATMENT PLANNING SYSTEMS AND METHODS FOR BODY CONTOURING APPLICATIONS”;

U.S. Patent Publication No. 2013/0158440 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Pat. No. 8,676,338 entitled “COMBINED MODALITY TREATMENT SYSTEMS, METHODS AND APPARATUS FOR BODY CONTOURING APPLICATIONS”;

U.S. Patent Publication No. 2014/0316393 entitled “COMBINED MODALITY TREATMENT SYSTEMS, METHODS AND APPARATUS FOR BODY CONTOURING APPLICATIONS”;

U.S. Pat. No. 8,603,073 entitled “SYSTEMS AND METHODS WITH INTERRUPT/RESUME CAPABILITIES FOR COOLING SUBCUTANEOUS LIPID-RICH CELLS”;

U.S. Pat. No. 9,737,434 entitled “SYSTEMS AND METHODS WITH INTERRUPT/RESUME CAPABILITIES FOR COOLING SUBCUTANEOUS LIPID-RICH CELLS”;

U.S. Pat. No. 8,702,774 entitled “DEVICE, SYSTEM AND METHOD FOR REMOVING HEAT FROM SUBCUTANEOUS LIPID-RICH CELLS”;

U.S. Pat. No. 9,861,520 entitled “DEVICE, SYSTEM AND METHOD FOR REMOVING HEAT FROM SUBCUTANEOUS LIPID-RICH CELLS”;

U.S. Patent Publication No. 2011/0300079 entitled “COMPOSITIONS FOR USE WITH A SYSTEM FOR IMPROVED COOLING OF SUBCUTANEOUS LIPID-RICH TISSUE”;

U.S. Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;

U.S. Pat. No. 6,041,787 entitled “USE OF CRYOPROTECTIVE AGENT COMPOUNDS DURING CRYOSURGERY”;

U.S. Pat. No. 6,032,675 entitled “FREEZING METHOD FOR CONTROLLED REMOVAL OF FATTY TISSUE BY LIPOSUCTION”;

U.S. Pat. No. 9,314,368 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Pat. No. 9,844,461 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Pat. No. 9,545,523 entitled “MULTI-MODALITY TREATMENT SYSTEMS, METHODS AND APPARATUS FOR ALTERING SUBCUTANEOUS LIPID-RICH TISSUE”;

U.S. Pat. No. 9,844,460 entitled “TREATMENT SYSTEMS WITH FLUID MIXING SYSTEMS AND FLUID-COOLED APPLICATORS AND METHODS OF USING THE SAME”;

U.S. Patent Publication No. 2015/0216720 entitled “TREATMENT SYSTEMS, METHODS, AND APPARATUSES FOR IMPROVING THE APPEARANCE OF SKIN AND PROVIDING FOR OTHER TREATMENTS”;

U.S. Pat. No. 9,861,421 entitled “COMPOSITIONS, TREATMENT SYSTEMS AND METHODS FOR IMPROVED COOLING OF LIPID-RICH TISSUE”;

U.S. patent Publication No. 2015/0216719 entitled “TREATMENT SYSTEMS AND METHODS FOR TREATING CELLULITE AND FOR PROVIDING OTHER TREATMENTS”;

U.S. patent application Ser. No. 14/662,181 entitled “TREATMENT SYSTEMS, DEVICES, AND METHODS FOR COOLING TARGETED TISSUE”;

U.S. patent application Ser. No. 14/710,407 entitled “TREATMENT SYSTEMS WITH ADJUSTABLE GAP APPLICATORS AND METHODS FOR COOLING TISSUE”;

U.S. Pat. No. 9,752,856 entitled “TREATMENT SYSTEMS, SMALL VOLUME APPLICATORS, AND METHODS FOR TREATING SUBMENTAL TISSUE”;

U.S. Patent Publication No. 2016/0051308 entitled “STRESS RELIEF COUPLINGS FOR CRYOTHERAPY APPARATUSES”;

U.S. Patent Publication No. 2016/0089550 entitled “TREATMENT SYSTEMS, METHODS, AND APPARATUSES FOR ALTERING THE APPEARANCE OF SKIN”;

U.S. Patent Publication No. 2017/0007309 entitled “TREATMENT SYSTEMS AND METHODS FOR AFFECTING GLANDS AND OTHER TARGETED STRUCTURES”;

U.S. Patent Publication No. 2016/0317346 entitled “SYSTEMS AND METHODS FOR MONITORING COOLING OF SKIN AND TISSUE TO IDENTIFY FREEZE EVENTS”;

U.S. Patent Publication No. 2017/0079833 entitled “TRANSCUTANEOUS TREATMENT SYSTEMS, COOLING DEVICES, AND METHODS FOR COOLING NERVES”;

U.S. Patent Publication No. 2017/0105869 entitled “VASCULAR TREATMENT SYSTEMS, COOLING DEVICES, AND METHODS FOR COOLING VASCULAR STRUCTURES”;

U.S. Patent Publication No. 2017/0196731 entitled “TEMPERATURE-DEPENDENT ADHESION BETWEEN APPLICATOR AND SKIN DURING COOLING OF TISSUE”;

U.S. Patent Publication No. 2017/0325992 entitled “SKIN FREEZING SYSTEMS FOR TREATING ACNE AND SKIN CONDITIONS”;

U.S. Patent Publication No. 2017/0325993 entitled “HYDROGEL SUBSTANCES AND METHODS OF CRYOTHERAPY”;

U.S. Patent Publication No. 2017/0326042 entitled “LIPOSOMES, EMULSIONS, AND METHODS FOR CRYOTHERAPY”;

U.S. Patent Publication No. 2017/0326346 entitled “PERMEATION ENHANCERS AND METHODS OF CRYOTHERAPY”; and

U.S. Patent Publication No. 2017/0239079 entitled “COOLING CUP APPLICATORS WITH CONTOURED HEADS AND LINER ASSEMBLIES.”

U.S. Patent Publication Nos. 2005/0251120 and 2008/0077211, and U.S. Pat. No. 8,285,390 are attached hereto as an Appendix, the entireties of which are hereby incorporated by reference herein and made a part of this application.

To the extent the foregoing commonly assigned U.S. patent applications and U.S. patents or any other material incorporated herein by reference conflicts with the present disclosure, the present disclosure controls.

### TECHNICAL FIELD

The present disclosure relates generally to treatment devices, systems, and methods for removing heat from subcutaneous lipid-rich tissue and/or other tissue. In particular, several embodiments are directed to cryoprotectant compositions, treatment systems, and methods for improved cooling of targeted tissue.

### BACKGROUND

Cooling treatments can be used to achieve aesthetic and/or therapeutic improvement of the human body, such as a reduction in excess adipose tissue (alternatively referred to as “body fat”). Excess body fat, or adipose tissue, may be present in various locations of the body, including, for example, the thigh, buttocks, abdomen, knees, back, face, arms, and other areas. For example, excess subcutaneous fat under the chin and/or around the neck can be cosmetically unappealing and, in some instances, can produce a “double chin.” A double chin can cause stretching and/or sagging of skin and may also result in discomfort. Excess adipose tissue can detract from personal appearance and athletic performance. Moreover, excess adipose tissue is thought to magnify the unattractive appearance of cellulite, which forms when subcutaneous fat lobules protrude or penetrate into the dermis and create dimples where the skin is attached to underlying structural fibrous strands. Cellulite and excessive amounts of adipose tissue are often considered to be cosmetically unappealing. Moreover, significant health risks may be associated with higher amounts of excess body fat.

Aesthetic improvement of the human body may involve the selective removal of adipose tissue. Invasive procedures (e.g., liposuction) for this purpose, however, tend to be associated with relative high costs, long recovery times, and increased risk of complications. Injection of drugs for reducing adipose tissue, such as submental or facial adipose tissue, can cause significant swelling, bruising, pain, numbness, and/or induration. Conventional non-invasive treatments for reducing adipose tissue may include regular exercise, application of topical agents, use of weight-loss drugs, dieting, or a combination of these treatments. One drawback of these non-invasive treatments is that they may not be effective or even possible under certain circumstances. For example, when a person is physically injured or ill, regular exercise may not be an option. Topical agents and orally administered weight-loss drugs are not an option if, as another example, they cause an undesirable reaction (e.g., an allergic or other negative reaction). Additionally, non-invasive treatments may be ineffective for selectively reducing specific regions of adiposity. For example, localized fat loss around the neck, jaw, cheeks, etc. often cannot be achieved using general or systemic weight-loss methods.

Other methods designed to reduce subcutaneous adipose tissue include laser-assisted liposuction and mesotherapy. Newer non-invasive methods include applying radiant energy to subcutaneous lipid-rich cells via, for example, radio frequency and/or light energy, such as described in U.S. Patent Publication No. 2006/0036300 and U.S. Pat. No. 5,143,063, or via, for example, high intensity focused ultrasound (HIFU) radiation, such as described in U.S. Pat. Nos. 7,258,674 and 7,347,855. Additional methods and devices for non-invasively reducing subcutaneous adipose tissue by cooling are disclosed in U.S. Pat. No. 7,367,341 entitled “METHODS AND DEVICES FOR SELECTIVE DISRUPTION OF FATTY TISSUE BY CONTROLLED COOLING” to Anderson et al. and U.S. Patent Publication No. 2005/0251120 entitled “METHODS AND DEVICES FOR DETECTION AND CONTROL OF SELECTIVE DISRUPTION OF FATTY TISSUE BY CONTROLLED COOLING” to Anderson et al., the entire disclosures of which are incorporated herein by reference.

Furthermore, aesthetic and/or therapeutic improvement of the human body may involve treatment or alteration of non-lipid-rich tissue as well as lipid-rich tissue, and again conventional treatments sometimes are not suitable for many subjects and cannot effectively target certain regions of tissue necessary for an effective treatment. For at least the foregoing reasons, there is a need for innovation in this field of aesthetic and/or therapeutic improvement of the human body.

### SUMMARY

In some embodiments, the present disclosure includes compositions for use with a system for freezing targeted cells at a site of a subject having skin. The compositions include at least a freezing point depressant configured to be applied to the skin of the subject at the site. The freezing point depressant lowers a freezing point of cells in an epidermal layer and/or a dermal layer of the skin. The compositions further include an anti-freeze protein configured to promote formation one or more needle-shaped ice crystals through the epidermal layer and/or dermal layers of the subject's skin, such that one or more needle-shaped ice crystals affect the cells. In some embodiments, the one or more needle-shaped ice crystals damage a membrane of one or more cells within the epidermal layer and/or dermal layer while the freezing point depressant inhibits damage to the epidermal layer and/or dermal layer.

In some embodiments, the composition is configured to be applied to the skin to generate a fractional freeze pattern in the skin of the site. The fractional freeze pattern is formed of one or more unfrozen zones and one or more frozen zones having a greater degree of freezing and/or damage to the cells within the zone compared to the unfrozen zones.

In some embodiments, the freezing point depressant lowers the freezing point of the cells in the epidermal and/or dermal layers to about −20° C. to about −5° C., about −18° C. to about −5° C., or about −15° C. to about −5° C.

In some embodiments, the composition includes one or more anti-freeze proteins selected from the group consisting of type I anti-freeze proteins, type II anti-freeze proteins, type III anti-freeze proteins, and anti-freeze glycoproteins.

In some embodiments, the anti-freeze protein is synthesized by solid-phase peptide synthesis and/or through recombinant expression in a host organism.

In some embodiments, the anti-freeze protein is derived from one or more organisms selected from the group consisting of a plant, a lichen, a fish, an insect, a bacterium, and a crustacean.

In some embodiments, the antifreeze protein is present in the composition in an amount of between about 1 μm/mL and about 100 mg/mL. In some embodiments, the needle-shaped crystals are less than or equal to about 10 μm in length along a c-axis. In some embodiments, the c-axis is longer than an a-axis and/or a b-axis, and the a-axis and b-axis are generally similar in length. The composition of any of the preceding examples, wherein the needle-shaped ice crystals are approximately 1000 times smaller than non-needle-shaped ice crystals along at least one axis. In some embodiments, the non-needle-shaped ice crystals enlarge along the a, b, and/or c-axis. In some embodiments, the fractional freeze pattern is formed by the alignment of one or more needle-shaped ice crystals along the c-axes.

In some embodiments, the present disclosure includes methods for affecting a subcutaneous layer of a human subject's body that include the steps of applying a freezing point depressant to a surface of the human subject's skin at a site; and freezing extracellular and/or intracellular water of a dermal layer and/or fat tissue in the human subject to form a fractional freeze pattern. The fractional freeze pattern is comprised of needle-shaped ice crystals through an epidermal and dermal layer by puncturing a cell membrane of a frozen cell within the site with the needle-shaped ice crystals to cause cell death.

In some embodiments, the freezing occurs in a targeted microzone.

In some embodiments, the cell death occurs via necrosis. In some embodiments, the necrosis occurs by a mechanical tearing of the cell membrane of the targeted cell by one or more needle-shaped ice crystals. In some embodiments, the cell death triggers a wound healing and/or inflammatory response. In some embodiments, the cell death initiates an increased production of collagen and/or elastin. In some embodiments, after the cell death, the subject exhibits skin that is more even and/or tighter compared to untreated skin.

In some embodiments, the subject exhibits minimal damage to the epidermal layer. In some embodiments, methods of the present disclosure further comprise triggering a healing and/or inflammatory response in the human subject's body.

In some embodiments, the freezing point depressant further includes at least one of a thickening agent, a pH buffer, a humectant, and a surfactant.

In some embodiments, the present disclosure includes systems for non-invasive, transdermal removal of heat from lipid-rich cells of a subject's body within a region. The system comprises a treatment unit, an applicator having a cooling unit in communication with the treatment unit, a freezing point depressant release structure formed of a carrier material and having an array of openings, and a freezing point depressant disposed within the carrier material of the freezing point depressant release structure.

In some embodiments, the array of openings includes at least four channels, each channel extending from a first channel opening at a first side of the release structure to second channel opening at a second side of the release structure to form continuous holes through the release structure. In some embodiments, the array of openings includes at least four chambers, each chamber having an opening at a first side of the release structure which extends into an interior of the release structure but not entirely through the release structure.

In some embodiments, the system is configured to deliver thermal treatment to the dermis and hypodermis.

In some embodiments, the region includes one or more target structures selected from the group consisting of sebaceous glands, sweat glands, collagen, elastin fibers, lipid-rich cells, and hair follicles.

In some embodiments, the freezing point depressant is configured to be applied to the skin to permeate into discrete portions of the skin to lower a freezing point of non-lipid-rich cells in the discrete portions of the skin.

In some embodiments, the release structure is configured to be positioned at the target region on a surface of the subject's skin. In some embodiments, the first side of the positioned release structure contacts the applicator. In some embodiments, the second side of the positioned release structure contacts the subject's skin. In some embodiments, the release structure is configured to retain and release the freezing point depressant between the surface of the applicator and the skin surface. The carrier material is an absorbent material and/or a microporous pad. In some embodiments, the release structure provides sustained release of the freezing point depressant to the skin surface at the lower temperature, and the lipid-rich cells in the target region are affected at the lower temperature while non-lipid-rich cells proximate the release structure are preserved. In some embodiments, the release structure provides sustained release of the freezing point depressant to the skin surface at the lower temperature. The dermal and hypodermal structures in the target region are affected at the lower temperature while epidermal structures proximate to the release structure are preserved.

In some embodiments, each opening of the array has a cross-sectional dimension of at least about 1 micrometer (μm). In some embodiments, each opening of the array is spaced apart from other openings of the array by at least about 1 μm. In some embodiments, an opening of the array has a cross-sectional area in a circular, elliptical, square, rectangular, triangular, or otherwise polygonal shape.

In some embodiments, the applicator is configured to reduce a temperature of the region beneath the epidermis of the subject selectively to reduce the temperature of lipid-rich cells in the target region from a natural body temperature to a lower temperature in the region and/or affect one or more structures within the region.

In some embodiments, the present disclosure includes methods for affecting a target region of a human subject's body that include the steps of positioning a freeze depressant release structure having an array of openings extending into a body of the freeze depressant release structure at a region on a surface of the human subject's skin, delivering a freezing point depressant having one or more anti-freeze proteins from a carrier material forming the freeze depressant release structure to a surface of skin at the region, and removing heat from the target region of the human subject to cool subcutaneous lipid-rich cells in the region to a temperature below normal body temperature.

In some embodiments, the array of openings includes at least four channels, each channel extending from a first channel opening at a first side of the release structure to second channel opening at a second side of the release structure to form continuous holes through the release structure. In some embodiments, the array of openings includes at least four chambers, each chamber having an opening at a first side of the release structure which extends into an interior of the release structure but not entirely through the release structure.

In some embodiments, removing heat from the region of the human subject further comprises removing heat from one or more portions of the region to cool one or more structures and/or one or more cells in the region. In some embodiments, the one or more portions of the region include the subject's dermal and/or hypodermal layers of the subject's skin. In some embodiments, the one or more other portions of the region include the subject's epidermal layer of the subject's skin. In some embodiments, removing heat from the region of the human subject further comprises creating one or more discrete partially frozen microchannels in the epidermal layer of the subject's skin which are separated by unfrozen areas of the epidermal layer. In some embodiments, removing heat from the region of the human subject generates a fractional freeze.

In some embodiments, the freezing point depressant is delivered to the region for about five minutes or less.

In some embodiments, during delivery of the freezing point depressant, a temperature of one or more target structures within one or more of the human subject's dermal structures is reduced.

In some embodiments, the one or more structures includes one or more sebaceous glands.

In some embodiments, positioning the release structure on the surface of the human subject's skin further comprises contacting the human subject's skin with a heat removal apparatus coupled to a portion of the release structure.

In some embodiments, contacting the human subject's skin with a heat removal apparatus coupled to a portion of the release structure further comprises removes heat from the human subject's epidermis without causing heat removal injury to the epidermis.

In some embodiments, delivering the freezing point depressant further comprises delivering a generally discontinuous and generally uniform amount of the freezing point depressant to the human subject's skin. In some embodiments, delivering the freezing point depressant further comprises delivering a generally continuous and generally non-uniform amount of the freezing point depressant to the human subject's skin.

In some embodiments, a portion of the region is contacted by a greater amount of the freezing point depressant compared to another portion of the region. In some embodiments, the portion of the region contacted by the greater amount of freezing point depressant freezes slower compared to another portion of the region.

In some embodiments, the anti-freeze proteins cause any water molecules that freeze to predominately freeze with a linear needle-like structure as opposed to a snowflake-like structure.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles may not be drawn to scale, and some of these elements are arbitrarily enlarged and positioned to improve drawing legibility. Further, the particular shapes of the elements as drawn are not intended to convey any information regarding the actual shape of the particular elements and have been solely selected for ease of recognition in the drawings.

FIG. 1 is a partially schematic, isometric view of a treatment system for non-invasively removing heat from subcutaneous lipid-rich target areas of a subject in accordance with an embodiment of the disclosure.

FIG. 1A illustrates an applicator of the treatment system of FIG. 1 cooling tissue in accordance with an embodiment of the disclosure.

FIG. 1B is a detailed view of an applicator-tissue interface of FIG. 1A illustrated by dashed lines labelled FIG. 1B in accordance with an embodiment of the disclosure.

FIG. 1C illustrates a fractional freeze pattern along a subject's skin in accordance with an embodiment of the disclosure.

FIG. 2 is a partially schematic, isometric view of an applicator suitable to be used with the treatment system of FIG. 1 in accordance with an embodiment of the disclosure.

FIG. 3 is a partially schematic, isometric view of another applicator suitable to be used with the treatment system of FIG. 1 in accordance with an embodiment of the disclosure.

FIG. 4A is a partially schematic, isometric view illustrating a freeze depressant release structure suitable to be used in the treatment system of FIG. 1 in accordance with yet another embodiment of the technology.

FIG. 4B is a partially schematic, isometric view illustrating a cross-section of the freeze depressant release structure of FIG. 4A in accordance with yet another embodiment of the technology.

FIG. 5A is a partially schematic, isometric view illustrating another freeze depressant release structure suitable to be used in the treatment system of FIG. 1 in accordance with yet another embodiment of the technology.

FIG. 5B is a partially schematic, isometric view illustrating yet another freeze depressant release structure suitable to be used in the treatment system of FIG. 1 in accordance with yet another embodiment of the technology.

FIG. 5C is a partially schematic, isometric view illustrating still another freeze depressant release structure suitable to be used in the treatment system of FIG. 1 in accordance with yet another embodiment of the technology.

FIG. 6A is a partially schematic, isometric view illustrating even another freeze depressant release structure suitable to be used in the treatment system of FIG. 1 in accordance with yet another embodiment of the technology.

FIG. 6B is a partially schematic, isometric view illustrating even yet another freeze depressant release structure suitable to be used in the treatment system of FIG. 1 in accordance with yet another embodiment of the technology.

FIG. 6C is a partially schematic, isometric view illustrating even still another freeze depressant release structure suitable to be used in the treatment system of FIG. 1 in accordance with yet another embodiment of the technology.

FIG. 7A is a partially schematic, isometric view illustrating another freeze depressant release structure suitable to be used in the treatment system of FIG. 1 in accordance with yet another embodiment of the technology.

FIG. 7B is a partially schematic, isometric view illustrating a cross-section of the freeze depressant release structure of FIG. 7A in accordance with yet another embodiment of the technology.

FIG. 7C is a partially schematic, isometric view of yet another freeze depressant release structure suitable to be used in the treatment system of FIG. 1 in accordance with yet another embodiment of the technology.

FIG. 7D is a partially schematic, isometric view of the embodiment of FIG. 7B applied to a patient in accordance with another embodiment of the technology.

FIG. 8A is a partially schematic, isometric view illustrating yet another freeze depressant release structure suitable to be used in the treatment system of FIG. 1 in accordance with yet another embodiment of the technology.

FIG. 8B is a partially schematic, isometric view illustrating a cross-section of the freeze depressant release structure of FIG. 8A in accordance with yet another embodiment of the technology.

FIG. 9 is a flow chart illustrating a method for cooling a treatment site in accordance with further embodiments of the invention.

FIG. 10 is a schematic block diagram illustrating computing system software modules and subcomponents of a computing device suitable to be used in the system of FIG. 1 in accordance with an embodiment of the technology.

### DETAILED DESCRIPTION

### A. Overview

The present disclosure describes treatment systems, freeze point depressant (e.g., cryoprotectant) release structures, freeze point depressant compositions, compounds for promoting the formation of one or more needle-shaped ice crystals, and methods for cooling of tissue in the treatment region using the same. In some embodiments, technologies of the present disclosure include cooling and/or freezing epidermal, dermal, hypodermal, and/or fat tissue (e.g., frozen tissue) while minimizing or limiting freeze-associated damage to other unfrozen tissue within the targeted area so as to achieve a fractional freezing type of effect. Without intending to be bound by any particular theory, it is thought that fractional freezing can reduce epidermal injury and/or any unwanted side effects (e.g., pigment changes and/or scarring) caused by thermal cooling and/or freezing of certain portions of the epidermal layer.

Freeze point depressants (e.g., freeze depressant) useful with the present technology can include one or more anti-freeze proteins which cause water molecules to freeze into a needle-like structure as opposed to a snowflake-like structure. Following application of the freeze point depressant to a subject's skin (e.g., treatment site) and freezing of certain water molecules, the resulting frozen needle-like structures can penetrate into cells of one or more layers of the subject's tissue at the treatment site, such as the dermal, hypodermal, and/or fat tissues. While the subject's skin is being cooled to a temperature at which ice crystals begin to form, the anti-freeze protein can bind to such ice crystals and regulate one or more propagation dimensions which generally results in shorter, sharper, more needle-like ice crystals compared to those formed in the absence of anti-freeze proteins. In this way, ice crystals formed by anti-freeze proteins are thought to cause more specific, discrete, and targeted damage to cells within the treatment site compared to ice crystals formed in the absence of anti-freeze proteins. If the cryoprotectant is applied to the skin in a non-uniform manner such that skin adjacent certain areas of the cryoprotectant does not freeze and other areas freeze, since the needle-like structures extend primarily vertically into the skin as opposed to horizontally, discrete columns of frozen tissue separated by unfrozen columns of tissue can be formed thereby resulting in a fractional freeze pattern (e.g., tissue subject to controlled freezing using the freeze point depressant surrounded by unfrozen tissue) within the treatment site. Unlike the frozen zones, the unfrozen zones experience limited ice-crystal formation overall and, when ice crystals do form, their shape is regulated by anti-freeze proteins which results in needle-like crystals. In addition to freeze point depressants, embodiments of the present technology can include freeze point depressant release structures having perforations (e.g., channels and/or chambers) that, when coupled to an applicator, deliver the freeze point depressant to the subject's skin in a non-uniform manner so that some areas of the epidermis will freeze while adjacent areas will not freeze or experience controlled, partial freezing in which needle-like ice crystals form. These freeze depressant release structures can also achieve fractional freezing by delivering the freeze point depressant to the subject's skin at a treatment site via a carrier material. According to one embodiment, the freeze point depressant is applied discontinuously so that some portions of the subject's skin are not contacted by any of the freeze point depressant release structure. Skin adjacent these areas not contacted by the freeze point depressant release structure readily freeze while areas of skin in contact with the freeze point depressant do not freeze to the same extent as areas of skin not in contact with the freeze point depressant (e.g., frozen zones) so as to generate the fractional freeze pattern. According to another embodiment, the freeze point depressant is applied onto the skin in a continuous but non-uniform manner (e.g., some areas have thicker and some areas thinner layers of freeze point depressant applied thereto). In this case, the skin areas adjacent the thinner areas of the freeze point depressant will preferentially freeze to again generate a fractional freeze pattern. The freeze point depressant and the freeze point depressant release structure can be used together, or separately. Without being bound by any particular theory, it is thought that freezing discrete portions of tissue surrounded by unfrozen tissue (e.g., fractional freezing) can result in a faster, more uniform healing response with less side effects when compared to uniform freezing methods that do not involve fractional freezing.

In some embodiments, a system for non-invasive, transdermal removal of heat from lipid-rich cells and/or structures within the dermis and/or hypodermis of a subject's body includes an applicator having a heat-exchanging element. The heat-exchanging element can be configured to reduce a temperature of a treatment region beneath the epidermis (e.g., a treatment region within the dermis and/or hypodermis) of the subject selectively to reduce the temperature of lipid-rich cells and/or structures within the dermis and/or hypodermis in the treatment region from a natural body temperature to a lower temperature in the treatment region. In one embodiment, the lower temperature can be less than −5° C. or −10° C., or in another embodiment between about −5° C. or −10° C. to about −15° C., or in another embodiment between about −5° C. or −10° C. −15° C. to about −25° C. The system can also include a freeze depressant (e.g., a cryoprotectant) configured to lower a freezing point of unfrozen cells and/or non-frozen structures in or near the frozen region. In one embodiment, the freeze depressant can be configured to lower the freezing point to about −20° C. to about −5° C. or −10° C., in another embodiment to about −18° C. to about −5° C. or −10° C., or in another embodiment to about −15° C. to about −5° C. or −10° C. In some embodiments, the freeze depressant can include one or more anti-freeze proteins which induce formation of needle-like ice crystals in one or more frozen cells. In one embodiment, the frozen region is subcutaneous adipose tissue (e.g., hypodermal layer). In another embodiment, the frozen region is in the dermal or epidermal layer of the subject's skin.

Other embodiments of the present technology include systems for non-invasively removing heat from lipid-rich cells, dermal structures, and/or hypodermal structures in a frozen region of a human subject's body. In one embodiment, the system can include an applicator having a cooling unit in communication with a treatment unit. The cooling unit is configured to reduce a temperature of the frozen region from a natural body temperature to a lower temperature in the frozen region.

The system can also include a freeze depressant release structure (e.g., cryoprotectant release structure) between a surface of the applicator and a skin surface in the frozen region. In some embodiments, the freeze depressant release structure is formed of a carrier material and includes an array of openings which can include channels (e.g., throughholes) and/or reservoirs. Channels extend from a first channel opening at a first side to a second channel opening at a second side whereas reservoirs are openings at a first and/or second side of the freeze depressant release structure but do not extend all the way through the release structure to form throughholes. In these embodiments, the freeze depressant release structure can be configured to retain a freeze depressant within the carrier material and release the freeze depressant between the surface of the applicator and the skin surface (e.g., epidermal layer) to one or more tissue regions during a tissue treatment procedure. For example, a portion of the epidermal layer in contact with the carrier material will not be frozen since the freeze point depressant is applied thereto, whereas a portion of the epidermal layer which is not in contact with the carrier material will be frozen since no freeze point depressant is applied thereto (e.g., this portion of the epidermis is in direct communication with the surface of the applicator via one or more channels and/or chambers of the freeze depressant release structure which is devoid of freeze point depressant material). By delivering the freeze depressant to the tissue portions to be frozen and not to tissue portions to be frozen, the system is configured to fractionally freeze (1) the epidermal and dermal layer of the treatment region and (2) microchannels of epidermal tissue in contact with the surface of the applicator (e.g., the frozen portion) can be created with the use of anti-freeze proteins which cause needle-shaped ice crystals to be formed. Without intending to be bound by any particular theory, it is thought that minimizing or eliminating delivery of the freeze depressant to the epidermis facilitates ice crystal inoculation and a generally shallow dermal freeze treatment while reducing epidermal thermal injury.

Compositions and formulations for use with devices and systems that enable tissue cooling (e.g., for alteration and reduction of adipose tissue, body contouring and augmentation, for the treatment of acne, for the treatment of hyperhidrosis, etc.), such as cryotherapy applications, are also described herein. In some embodiments, these compositions and formulations protect non-frozen cells, such as non-lipid-rich cells (e.g., in the dermal and epidermal skin layers) and/or non-frozen structures, by preventing or limiting freeze damage during dermatological and related aesthetic procedures that require sustained exposure to cold temperatures. Embodiments of the disclosure are further directed to methods, compositions and devices that provide controlled freezing to dermal and/or fat tissue while minimizing damage to epidermal tissue by forming a fractional freezing pattern through the epidermal layer and/or dermal layer. For example, the compositions and formulations can include one or more anti-freeze proteins which provide controlled and discrete damage to the cells of the dermal layer and fat tissue by freezing the intracellular and extracellular water into needle-shaped ice crystals. The needle-shaped ice crystals can puncture the cell membrane of the frozen cells comprising the dermal layer and fat tissue, triggering controlled cell damage and in some embodiments of the disclosure, controlled cell death. In some embodiments of the disclosure, cell death occurs via necrotic cell death. Further embodiments of the disclosure include systems for controlled cell death and/or cell damage, which further triggers a healing and/or inflammatory response. This healing and/or inflammatory response initiates an increased production of collagen and/or elastin, which in turn further provides even and tightened skin.

Various embodiments of the technology are directed to compositions for use with a system for cooling subcutaneous lipid-rich tissue of a subject's skin and/or altering one or more structures disposed within the subject's dermis and/or hypodermis. In one embodiment, the composition can include a freeze point depressant (e.g., a cryoprotectant) and an anti-freeze protein and is configured to be applied to the skin of the subject. The freeze point depressant can be configured to lower a freezing point of cells and/or structures in a epidermal layer and/or a dermal layer of the skin and can facilitate formation of certain types of ice crystals using anti-freeze protein-mediated inoculation. In some embodiments, the freeze point depressant can include one or more anti-freeze proteins which provide a discrete injury of the cells comprising the subject's dermis and/or fat tissue. For example, the anti-freeze proteins produce a thermal hysteresis (e.g., create a difference in the melting and freezing points of the cells in an epidermal layer and/or a dermal layer) upon binding to the ice crystal surface. However, unlike a conventional cryoprotectant, compositions including a freeze point depressant and one or more anti-freeze proteins do not necessarily lower the freezing point of the cells in an epidermal layer and/or a dermal layer of the skin. Rather, as a function of the concentration of the anti-freeze proteins present (e.g., the anti-freeze proteins have non-colligative properties), the anti-freeze proteins within the freeze point depressant control the types of ice crystals that form in the epidermal layer and/or the dermal layer. By doing so, freeze point depressant and/or freeze point depressant release structures configured in accordance with the present technology can generate a fractional freezing pattern which prevents excessive epidermal damage conventionally associated with removing heat from a tissue using a bulk freeze approach. In addition, the freeze point depressant can also include at least one of a thickening agent, a pH buffer, a humectant, and a surfactant. For example, the composition can include one or more thickening agents, one or more pH buffers, one or more humectants, and/or one or more surfactants.

In one embodiment, the frozen cells have a higher lipid content than the non-frozen cells. For example, the frozen cells can be subcutaneous lipid-rich cells. In another embodiment, the frozen cells are cells associated with exocrine glands within or near the skin (e.g., epidermal and/or dermal layers) of a subject. For example, the frozen cells may be lipid-producing cells residing within or at least proximate to sebaceous glands, or in another embodiment, apocrine sweat glands. In other embodiments, the target structures include collagen fibers, elastin fibers, and hair follicles.

In some embodiments, a treatment composition for application to a treatment site can include one or more freeze depressants (e.g., cryoprotectants) and one or more anti-freeze proteins. The freeze depressant can protect some tissue from freeze injuries, and the anti-freeze protein can be an ice crystal limiter, an ice crystal morphology controller, or other type of composition for controlling ice crystal formation, growth, or the like. Anti-freeze proteins can be configured to inhibit or limit ice crystal size (e.g., length, width, etc.), distribution, or combinations thereof. For example, anti-freeze proteins can promote the formation of ice crystals having particular shapes. In some embodiments, anti-freeze proteins can promote the formation of slender or needle-shaped ice crystals. As explained in greater detail below, anti-freeze proteins are referred to as such due to its biological nomenclature in its native state when found within certain organisms. In some embodiments, the freeze depressant is a cryoprotectant for reducing the freezing point of non-targeted tissue, cells, or structures, and can optionally include one or more anti-freeze proteins. The freeze point depressant and anti-freeze proteins can be configured to affect different tissue. For example, anti-freeze proteins can allow ice crystal formation in targeted tissue while the freeze point depressant protects non-targeted tissue. This allows for the formation of discrete freezing zones with well-defined boundaries suitable for producing fractional freezing patterns within, for example, a targeted layer of tissue.

Further embodiments of the present technology are directed to treatment methods for affecting a region of a human subject's body to alter subcutaneous adipose tissue, dermal structures, and/or hypodermal structures. In one embodiment, a method can include positioning a freeze depressant release structure having one or more channels or extending through a body of the freeze depressant release structure at a region on a surface of the human subject's skin, delivering a cryoprotectant having one or more anti-freeze proteins from a material forming the freeze depressant release structure to a surface of skin at a site, and removing heat from a frozen region of the human subject to cool subcutaneous lipid-rich cells in the frozen region to a temperature below normal body temperature. In other embodiments, the release structure includes one or more chambers rather than one or more channels.

The method can also include removing heat from one or more portions of the region to cool one or more target structures and/or one or more target cells in the region. In other embodiments, the method can include positioning a freeze depressant release structure, having one or more chambers disposed on either or both side or sides of the freeze depressant release structure. In other embodiments, channels can replace the chambers. In some embodiments, during delivery of the freeze depressant, a temperature of one or more structures within one or more of the human subject's dermal structures is reduced. Additional methods for affecting a region of a subject's body can include contacting the human subject's skin with a heat removal apparatus coupled to a portion of the freeze depressant release structure, removing heat from the human subject's epidermis without causing heat removal injury to the epidermis, and/or delivering a generally discontinuous and generally uniform amount of the freeze depressant to the human subject's skin.

In some embodiments, the system can further include a freeze depressant configured to lower a freezing point of non-targeted cells (e.g., non-lipid-rich cells) in or near the region. In one embodiment, the freeze depressant can include one or more anti-freeze proteins which provide controlled and discrete damage to the cells of the dermal layer and fat tissue by freezing the intracellular and/or extracellular water of the dermal layer and fat tissue at a targeted microzone. Without intending to be bound by any particular theory, freezing the targeted microzone enables a faster recovery than conventional cryolipolysis which causes bulk tissue freezing by reducing damage of unfrozen cells and/or tissue by having areas of frozen tissue separated by areas of unfrozen tissue. The composition can further include one or more anti-freeze proteins which can facilitate generation of needle-shaped ice crystals that enhance a fractional freeze pattern through the epidermal layer and/or dermal layer by limiting ice crystal formation to mostly a vertical inward direction as opposed to a transverse direction generally parallel to a skin surface. In some embodiments, the composition is configured to trigger a healing and/or inflammatory response that may be attributed to necrotic cell death. Without intending to be bound by any particular theory, it is thought that the healing and/or inflammatory response initiates an increased production of collagen and/or elastin and as a result, provides even and tightened skin compared to conventional cryoprotectants.

In another embodiment, a system for non-invasive, transdermal removal of heat from lipid-rich cells of a subject's body includes an applicator having a heat-exchanging element. The heat-exchanging element can be configured to reduce a temperature of a region beneath the epidermis of the subject selectively to reduce the temperature of lipid-rich cells in the target region from a natural body temperature to a lower temperature in the target region. In one embodiment, the lower temperature can be less than −5° C. or −10° C., or in another embodiment between about −5° C. or −10° C. to about −15° C., or in another embodiment between about −5° C., −10 C or −15° C. to about −25° C. The system can also include a first freeze depressant configured to lower a freezing point of non-frozen cells in or near the region. In one embodiment, the first freeze depressant can be configured to lower the freezing point of the cells to about −20° C. to about −5° C. or −10° C., in another embodiment to about −18° C. to about −5° C. or −10° C., or in another embodiment to about −15° C. to about −5° C. or −10° C. In one embodiment, the freeze depressant can include one or more of anti-freeze proteins which induce formation of needle-shaped ice crystals formed from intracellular and/or extracellular water of the dermal layer and fat tissue, thereby creating a fractional freeze pattern through the epidermal layer and/or dermal layer at a targeted microzone region. The freeze depressant comprising the anti-freeze proteins, in some embodiments, protects cells and tissues not in the frozen microzone when the needle-shaped ice crystals are formed. In one embodiment, the region is subcutaneous adipose tissue. In another embodiment, the region is in the dermal layer of the subject's skin.

Further embodiments of the present technology are directed to treatment methods for affecting a region of a human subject's body to alter subcutaneous adipose tissue. In another embodiment, methods of the present technology include applying a freeze depressant having one or more anti-freeze proteins to a surface of the skin at a treatment site followed by freezing the site. In another embodiment, freeze depressants of the present technology can induce the formation of needle-shaped crystals that puncture the cell membrane of the targeted cells, initiating necrotic cell death. In one embodiment, necrotic cell death is characterized by ice crystal-induced mechanical tearing of the cell membrane. Without intending to be bound by any particular theory, it is thought that necrotic cell death induces a wound healing and/or inflammatory response.

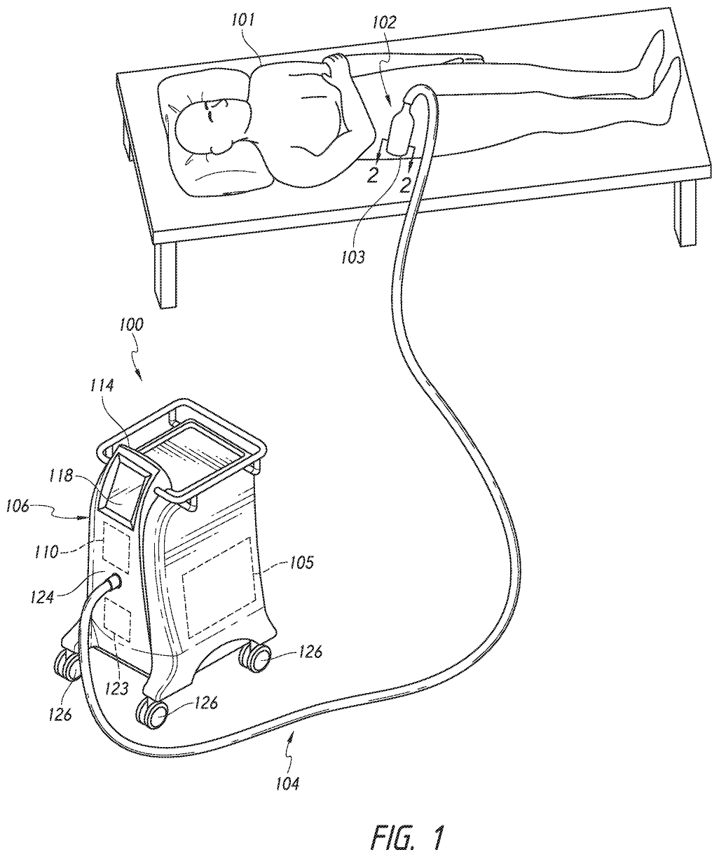

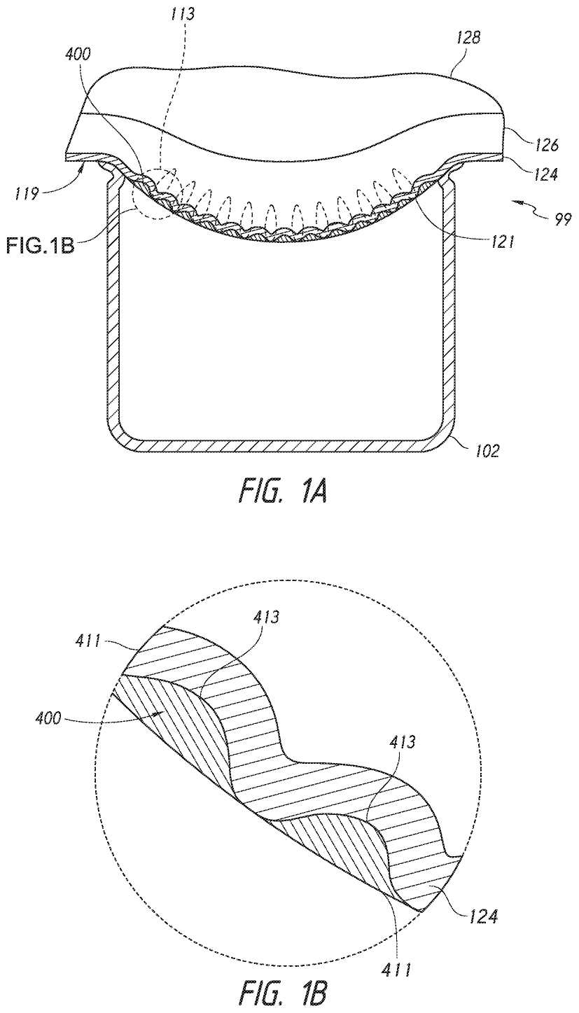

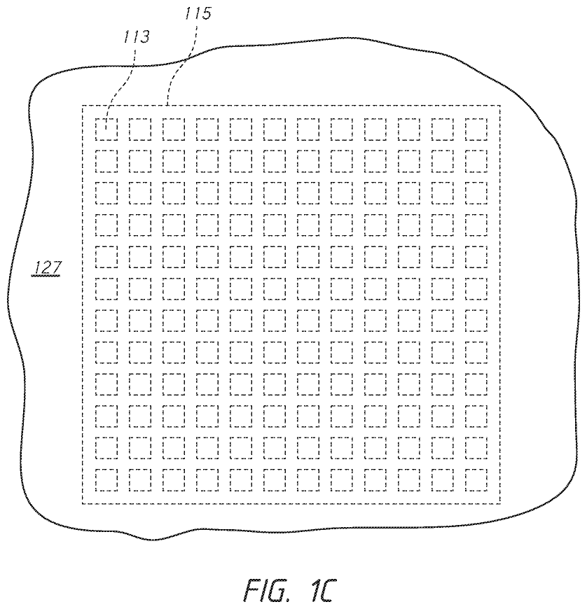

Specific details of methods for cooling tissue and related structures and systems in accordance with several embodiments of the present invention are described herein with reference to FIGS. 1-10. Although methods for cooling tissue and related structures and systems may be disclosed herein primarily or entirely in the context of cryolipolysis and cryolysis, other contexts in addition to those disclosed herein are within the scope of the present invention. For example, the disclosed methods, structures, and systems may be useful in the context of any compatible type of treatment mentioned in the applications and patents listed above and incorporated herein by reference. It should be understood, in general, that other methods, structures, and systems in addition to those disclosed herein are within the scope of the present invention. For example, methods, structures, and systems in accordance with embodiments of the present invention can have different and/or additional configurations, components, and procedures than those disclosed herein. Moreover, a person of ordinary skill in the art will understand that methods, structures, and systems in accordance with embodiments of the present invention can be without one or more of the configurations, components, and/or procedures disclosed herein without deviating from the present invention.

For ease of reference, saccharides and saccharide derivatives (i.e., modified saccharides) may be collectively referred to as “saccharides” in this disclosure. Furthermore, the term “saccharides” in this disclosure should be considered to encompass natural saccharides, artificial saccharides, and other saccharide-like polyhydroxy aldehydes and ketones. The term “treatment system,” as used generally herein, refers to cosmetic, therapeutic, or other medical treatment systems, as well as to any associated treatment regimens and medical device usages. At least some treatment systems configured in accordance with embodiments of the present invention are useful for reducing or eliminating excess adipose, other undesirable tissue, or undesirable structures, and/or for enhancing the appearance of skin. In many cases, the treatment systems can be used at various locations, including, for example, a subject's face, neck, abdomen, thighs, buttocks, knees, back, arms, and/or ankles. The term “tissue,” as used generally herein, may refer to a region of cells and associated extracellular material or to a type of cells and associated extracellular material.

Treatment systems in accordance with at least some embodiments of the present invention are well suited for cosmetically beneficial alterations of tissue at targeted anatomical regions. Some cosmetic procedures may be for the sole purpose of altering a target region to conform to a cosmetically desirable look, feel, size, shape, and/or other desirable cosmetic characteristic or feature. Accordingly, at least some embodiments of the cosmetic procedures can be performed without providing an appreciable therapeutic effect (e.g., no therapeutic effect). For example, some cosmetic procedures may not include restoration of health, physical integrity, or the physical well-being of a subject. The cosmetic methods can target subcutaneous or dermal regions to change a subject's appearance and can include, for example, procedures performed on subject's submental region, face, neck, ankle region, or the like. In other embodiments, however, desirable treatments may have therapeutic outcomes, such as alteration of vascular malformations, treatment of glands including sebaceous and sweat glands, treatment of nerves, alteration of body hormones levels (by the reduction of adipose tissue), etc.

Some of the embodiments disclosed herein can be for cosmetically beneficial alterations of a variety of body regions. As such, some treatment procedures may be for the sole purpose of altering the body region to conform to a cosmetically desirable look, feel, size, shape, or other desirable cosmetic characteristic or feature. Accordingly, at least some embodiments of the cosmetic procedures can be performed without providing any, or in another embodiment, providing minimal therapeutic effect. For example, some treatment procedures may be directed to treatment goals that do not include restoration of health, physical integrity, or the physical well-being of a subject. In other embodiments, however, the cosmetically desirable treatments may have therapeutic outcomes (whether intended or not), such as, psychological benefits, alteration of body hormones levels (by the reduction of adipose tissue), etc. The cosmetic methods can target subcutaneous regions to change a subject's appearance such as, for example, procedures performed on a subject's “love-handles” (i.e., excess adipose tissue at the side of a subject's waistline). In another embodiment, the cosmetic methods can target sebaceous glands in the subject's skin to change a subject's appearance such as, for example, procedures performed on a subject's face. In another embodiment, the cosmetic methods can target sweat glands in the subject's skin to treat hyperhidrosis.

Several of the details set forth below are provided to describe the following examples and methods in a manner sufficient to enable a person skilled in the relevant art to practice, make, and use them. Several of the details and advantages described below, however, may not be necessary to practice certain examples and methods of the technology. Additionally, the technology may include other examples and methods that are within the scope of the technology but are not described in detail.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the technology. The headings provided herein are for convenience only and are not intended to limit or interpret the scope or meaning of the technology.

### B. Cryotherapy

FIG. 1 and the following discussion provide a brief, general description of a treatment system 100 in accordance with some embodiments of the technology. The treatment system 100 can be a temperature-controlled system for exchanging heat with a subject 101 and can include a non-invasive tissue-cooling apparatus in the form of a cooling cup applicator 103 (“applicator 103”) configured to selectively cool tissue to affect targeted tissue, structures, or the like. The illustrated applicator 103 is positioned along a subject's hip and can draw a vacuum to provide suitable thermal contact with the subject's skin to cool subcutaneous adipose tissue. The applicator 103 is configured to facilitate a high amount of thermal contact with the subject's skin by minimizing, limiting, or substantially eliminating air gaps at the applicator/tissue interface. The entire skin surface of the retained volume of tissue can be cooled for efficient treatment. The applicator 103 can have a relatively shallow tissue-receiving chamber to avoid or limit pooling of blood, rupturing of blood vessels, patient discomfort, and so forth.

The applicator 103 can be used to perform medical treatments to provide therapeutic effects and/or cosmetic procedures for cosmetically beneficial effects. Without being bound by theory, selective effects of cooling are believed to result in, for example, membrane disruption, cell shrinkage, disabling, disrupting, damaging, destroying, removing, killing, and/or other methods of cell and/or tissue alteration. Such alteration is believed to stem from one or more mechanisms acting alone or in combination. It is thought that such mechanism(s) trigger an apoptotic cascade, which is believed to be the dominant form of cell death and structural damage caused by non-invasive cooling. In any of these embodiments, the effect of tissue cooling can be the selective reduction of cells by a desired mechanism of action, such as apoptosis, lipolysis, or the like. In some procedures, the applicator 103 can cool the skin surface and/or targeted tissue to cooling temperature in a range of from about −25° C. to about 20° C. In other embodiments, the cooling temperatures can be from about −20° C. to about 10° C., from about −18° C. to about 5° C., from about −15° C. to about 5° C., or from about −15° C. to about 0° C. In further embodiments, the cooling temperatures can be equal to or less than −5° C., −10° C., −15° C., or, in yet another embodiment, from about −15° C. to about −25° C. Other cooling temperatures and temperature ranges can be used.

Apoptosis, also referred to as “programmed cell death”, is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990).

One embodiment of apoptosis, in contrast to cellular necrosis (a non-ordered form of cell death causing local inflammation), is that apoptotic cells express and display phagocytic markers on the surface of the cell membrane, thus marking the cells for phagocytosis by macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption or dysfunction, thereby inducing apoptosis. This mechanism is well-documented for many cell types and may be active when adipocytes, or lipid-rich cells, are cooled. Mazur, P., “Cryobiology: The Freezing of Biological Systems” Science, 68: 939-949 (1970); Quinn, P. J., “A Lipid Phase Separation Model of Low Temperature Damage to Biological Membranes” Cryobiology, 22: 128-147 (1985); Rubinsky, B., “Principles of Low Temperature Preservation” Heart Failure Reviews, 8, 277-284 (2003). Other possible mechanisms of adipocyte damage, described in U.S. Pat. No. 8,192,474, relate to ischemia/reperfusion injury that may occur under certain conditions when such cells are cooled as described herein. For instance, during treatment by cooling as described herein, the targeted adipose tissue may experience a restriction in blood supply and thus be starved of oxygen due to isolation as a result of applied pressure, cooling which may affect vasoconstriction in the cooled tissue, or the like. In addition to the ischemic damage caused by oxygen starvation and the buildup of metabolic waste products in the tissue during the period of restricted blood flow, restoration of blood flow after cooling treatment may additionally produce reperfusion injury to the adipocytes due to inflammation and oxidative damage that is known to occur when oxygenated blood is restored to tissue that has undergone a period of ischemia. This type of injury may be accelerated by exposing the adipocytes to an energy source (e.g., via thermal, electrical, chemical, mechanical, acoustic, or other means) or otherwise increasing the blood flow rate in connection with or after cooling treatment as described herein. Increasing vasoconstriction in such adipose tissue by, for example, various mechanical means (e.g., application of pressure or massage), chemical means or certain cooling conditions, as well as the local introduction of oxygen radical-forming compounds to stimulate inflammation and/or leukocyte activity in adipose tissue may also contribute to accelerating injury to such cells. Other yet-to-be understood mechanisms of injury may exist.

Necrotic cell death is generally referred to as “unregulated cell death.” Necrosis is caused by external factors, such as an infection, toxin, and/or trauma, which induce a series of biochemical events that ultimately lead to a rupturing of the cell membrane and subsequent release of the intracellular contents to the extracellular matrix. These changes include swelling of the cell's organelles, structural modifications to the nucleus, chromatin condensation into small, irregular fragments, and increased cell volume via oncosis, which destroy the cell membrane. A freeze-thaw injury via an external stimulus, such as cold exposure is one mechanism that can induce cellular necrosis. Golstein, P., Guido, K. “Cell Death by Necrosis: towards a molecular definition” Trends in Biomedical Sciences 32, 37-43 (2007); Karch, J.; Molkentin, J. D. “Regulated Necrotic Cell Death” Circulation Research, 1800-1810 (2018).

Unlike apoptosis, necrosis does not cause cell fragmentation into discrete apoptotic bodies but rather releases cellular contents into the surrounding extracellular matrix. This release of cellular contents is thought to induce an inflammatory response in the surrounding tissue, which attracts leukocytes and phagocytes. Rock, K. L.; Kono, H. “The Inflammatory Response to Cell Death” Annual Review Pathology 3, 99-126 (2008).

In addition to the apoptotic and necrotic mechanisms involved in lipid-rich cell death, local cold exposure is also believed to induce lipolysis (i.e., fat metabolism) of lipid-rich cells and has been shown to enhance existing lipolysis which serves to further increase the reduction in subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

One expected advantage of the foregoing techniques is that the subcutaneous frozen cells in the region can be reduced generally without collateral damage to non-frozen cells in the same region. In addition, lipid-rich cells can be affected at low temperatures that do not affect non-lipid-rich cells. As a result, lipid-rich cells, such as those associated with highly localized adiposity (e.g., adiposity along the abdomen, submental adiposity, submandibular adiposity, facial adiposity, etc.), can be affected while non-lipid-rich cells (e.g., myocytes) in the same generally region are not damaged. The unaffected non-lipid-rich cells can be located underneath lipid-rich cells (e.g., cells deeper than a subcutaneous layer of fat), in the dermis, in the epidermis, and/or at other locations.

In some procedures, the treatment system 100 can remove heat from underlying tissue through the upper layers of tissue and create a thermal gradient with the coldest temperatures near the cooling surface, or surfaces, of the applicator 103 (i.e., the temperature of the upper layer(s) of the skin can be lower than that of the targeted underlying target cells). It may be challenging to reduce the temperature of the targeted cells low enough to be destructive to these target cells (e.g., induce apoptosis, cell death, etc.) while also maintaining the temperature of the upper and surface skin cells high enough so as to be protective (e.g., non-destructive). The temperature difference between these two thresholds can be small (e.g., approximately, 5° C. to about 10° C., less than 10° C., less than 15° C., etc.). Protection of the overlying cells (e.g., typically water-rich dermal and epidermal skin cells) from freeze damage during dermatological and related aesthetic procedures that involve sustained exposure to cold temperatures may include improving the freeze tolerance and/or freeze avoidance of these skin cells by using, for example, freeze depressants (e.g., cryoprotectants) for inhibiting or preventing such freeze damage.

Tissue can be rapidly rewarmed as soon as practicable after a freeze event has occurred to limit, reduce, or prevent damage and adverse side effects associated with the freeze event. After freezing begins, tissue can be rapidly warmed as soon as possible to minimize or limit damage to tissue, such as the epidermis. In some procedures, tissue is partially or completely frozen for a predetermined period of time and then warmed. According to one embodiment, an applicator can warm shallow tissue using, for example, thermoelectric elements in the device. Thermoelectric elements can include Peltier devices capable of operating to establish a desired temperature (or temperature profile) along the surface. In other embodiments, the applicator outputs energy to warm tissue. For example, the applicator can have electrodes that output radiofrequency energy for warming tissue. In some procedures, the tissue can be warmed at a rate of about 1° C./s, 2° C./s, 2.5° C./s, 3° C./s, 5° C./s, or other rate selected to thaw frozen tissue after the tissue has been partially or completely frozen for about 10 seconds, 30 seconds, 1 minute, 2 minutes, 5 minutes, 10 minutes, or other suitable length of time.

In some embodiments, the treatment system 100 can cool the skin of the patient to a temperature in a range of from about −20° C. to about 20° C. In other embodiments, the cooling temperatures can be from about −20° C. to about 10° C., from about −18° C. to about 5° C., from about −15° C. to about 5° C., or from about −15° C. to about 0° C. In further embodiments, the cooling temperatures can be less than −10° C., or in yet another embodiment, from about −15° C. to about −25° C.

As explained in more detail below, a freeze depressant having a freezing point in the range of about −40° C. to about 0° C. can be applied to the surface of the skin of the patient or subject 101, or to an interface between the treatment device or applicator 103 and the skin of the patient or subject 101. As used herein, “cryoprotectant,” “cryoprotectant agent,” and “cryoprotective” mean substances (e.g., compositions, formulations, compounds, etc.) that assist in preventing freezing of unfrozen tissue (e.g., dermal and/or epidermal tissue) compared to an absence of the substance(s). In some embodiments, the cryoprotectant does not include any anti-freeze proteins.

Further, the freeze depressant can also enable the treatment device or applicator to be maintained at a desired temperature while preventing ice from forming on a surface of the treatment device or applicator, and thus reduces the delay in reapplying the treatment device or applicator to the subject. Yet another embodiment of the technology is that the freeze depressant may prevent the treatment device or applicator from freezing to the skin of the patient or subject. Additionally, the freeze depressant may protect biological tissues of a subject, such as a mammal, from freezing damage (e.g., damage due to ice formation). The freeze depressant composition may also include one or more additives present in the compound and configured to provide selected properties to the compound. Further details regarding freeze depressants (e.g., cryoprotectants) suitable for use with the treatment system and/or in treatment regimens associated with cooling tissue, dermal, and/or hypodermal structures are described in greater detail below.

### C. Suitable Treatment System

Referring to FIG. 1, the illustration is a partially schematic, isometric view showing one example of the treatment system 100 for non-invasively removing heat from subcutaneous lipid-rich target areas of the patient or subject 101, such as an abdominal area 102 or another suitable area. The applicator 103 can engage the target area of the subject 101 and a treatment unit 106 that operate together to cool or otherwise remove heat from the subcutaneous lipid-rich cells of the subject 101. The applicator 103 can be part of an application system, and the applicator 103 can have various configurations, shapes, and sizes suitable for different body parts such that heat can be removed from any cutaneous or subcutaneous lipid-rich target area of the subject 101. For example, various types of applicators may be applied during treatment, such as a vacuum applicator, a belt applicator (either of which may be used in combination with a massage or vibrating capability), and so forth. Each applicator 103 may be designed to treat identified portions of the patient's body, such as chin, cheeks, arms, pectoral areas, thighs, calves, buttocks, abdomen, “love handles”, back, breast, and so forth.

FIG. 1A is a schematic cross-sectional view of the applicator 103 cooling a region in accordance with an embodiment of the disclosure. FIG. 1B is a detailed view of the applicator-tissue interface of FIG. 1A. While not intending to be limiting, the freeze depressant release structure 400 illustrated in FIGS. 1A and 1B includes chambers which are discussed in greater detail in reference to FIGS. 7A-7D.

Referring now to FIG. 1A, an applicator device 99 can include an applicator 102 (internal components not shown) and a freeze depressant carrier in the form of a freeze depressant release structure 400. The applicator 102 can include a temperature-controlled surface 121 for absorbing heat to cool, for example, the epidermis 124, dermis 126, hypodermis/subcutaneous tissue 128, and/or freezing other structures. The freeze depressant release structure 400 (shown in cross-section) can deliver a freeze depressant (e.g., cryoprotectant) that protects non-frozen tissue while allowing controlled freezing (including partial freezing) of frozen tissue or structures. The freeze depressant release structure 400 also produces fractional cooling of tissue for forming a fractional freeze pattern at the region. In use, when downward pressure is applied to the applicator 102 after being placed at the region, the freeze depressant release structure 400 and skin 124 will compress together thereby eliminating any air gaps between the skin 124 and the temperature-controlled surface 121. Once the freeze depressant release structure 400 and skin 124 have been compressed together, a cross-sectional dimension of the compressed freeze depressant release structure 400 differs from that of an uncompressed freeze depressant release structure 400. As illustrated in FIG. 1A (and shown in the enlarged view in FIG. 1B), the compressed freeze depressant release structure 400 includes at least three cross-sectional dimensions, a first greater than a second which is greater than a third (e.g., where the skin 124 contacts the temperature-controlled surface 121). The second cross-sectional dimension provides less freeze protection than the thicker first cross-sectional dimension since less cryoprotectant is present at the second cross-sectional dimension. It is expected that a first amount of unfrozen cells, tissues, and/or structures is greatest at or near the first cross-sectional dimension, a second amount of unfrozen cells, tissues, and/or structures is less than the first amount at or near the second cross-sectional dimension, and a third amount of unfrozen cells, tissues, and/or structures is absent at or near the third cross-sectional dimension (e.g, the cells, tissues, and/or structures at or nar the third cross-sectional dimension are frozen).

The freeze depressant release structure 400 can be a flexible pad impregnated with a freeze depressant composition (e.g., a gel) configured to contact the subject's skin surface 119 without flowing along the skin surface 119. The isolated gel freeze depressant can help produce the spaced apart frozen zones 113 (one identified) which freeze when contacting the applicator 103. In procedures, most of the shallow tissue directly underneath the freeze depressant release structure 400 is protected while the frozen zones 113 underneath the openings (e.g., channels and/or chambers) can be frozen.

The freeze depressant can lower the freezing point of the subject's non-frozen tissue. For example, the freeze depressant can include a cryoprotective substance that lowers a freezing point of cells in the epidermal and/or dermal layer. The freeze depressant can also include one or more anti-freeze proteins (e.g., ice crystal growth inhibitors) that control the morphology of ice crystals that form in any tissue that is frozen. For example, anti-freeze proteins can allow relatively small needle-shaped ice crystals to form in epidermal and/or dermal. Additionally, small needle-shaped ice crystals can localize tissue injury, thereby enabling a wide range of freeze patterns.

The composition of the freeze depressant can be selected based on the location, type, and/or characteristics of the frozen tissue. In some procedures for selectively reducing simultaneous fat, a topically applied temperature point depressant can protect the epidermal and/or dermal layers. Delivery of the freezing point depressant can be localized to the skin to allow freezing of the subcutaneous fat. The anti-freeze proteins may be delivered through the skin to the subcutaneous tissue to control localized freezing (including partial freezing) in the subcutaneous tissue. In some procedures for selectively altering the dermal layer, the freezing point depressant can be delivered to the subject's skin to protect the epidermal layer and the needle-like crystals form on the subject's epidermal surface. Once formed, the needle-like crystals extend into the subject's dermis. The region can be cooled to cause localized freezing in the dermal layer. In both procedures, the temperature point depressant can protect the epidermis from freeze injury.

FIG. 1B is a detailed view of the applicator-tissue interface of FIG. 1A in accordance with an embodiment of the disclosure. The freeze depressant release structure 400 can define windows 411 that allow thermal contact between the temperature-controlled surface 121 and the skin surface 127. FIG. 1B shows the skin 124 protruding through the openings 411 to directly contact the temperature-controlled surface 121 for efficient heat transfer. As explained with reference to FIG. 1A, the freeze depressant release structure compresses when in use (e.g., when in contact with the skin 124 and the applicator 102 and downward pressure is applied). The freeze depressant release structure 400 has sections 413 between the openings 411, and those sections 413 can release cryoprotectant so that epidermal tissue in contact with it does not freeze. The non-uniform distribution of the cryoprotectant produces the freeze pattern shown in FIG. 1C.

FIG. 1C illustrates a fractional freeze pattern at the site in accordance with an embodiment of the disclosure. The fractional freeze pattern includes a unfrozen zone 115 (one identified) and the spaced apart frozen zones 113 (one identified). As explained above with reference to FIG. 1B, the unfrozen zones 115 represent portions of the skin surface that were contacted by the freeze depressant release structure 400. The frozen zones 113 can be round, elliptical shaped, or polygonal-shaped (including rounded polygonal) zones a dimension (e.g., width, height, etc.) equal to or less than about 8 mm, 7 mm, 6 mm, 5 mm, 4 mm, 3 mm, 2 mm, 1 mm, 0.5 mm, 0.1 mm, 90 μm, 80 μm, 70 μm, 60 μm, 50 μm, 40 μm, 30 μm, 20 μm, 10 μm, 5 μm, 1 μm or less than 1 μm and have depths of about 8 mm, 7 mm, 6 mm, 5 mm, 4 mm, 3 mm, 2 mm, 1 mm, 0.5 mm, 0.1 mm, 90 μm, 80 μm, 70 μm, 60 μm, 50 μm, 40 μm, 30 μm, 20 μm, 10 μm, 5 μm, 1 μm or less than 1 μm. The depths can be varied by varying the treatment time and treatment temperature. A desired depth will depend on what tissue or tissue structure is desired to be affected. For example, frozen zones 113 for affecting subcutaneous fat can be deeper than unfrozen zones 115 for affecting glands, hair follicles, or other shallow structures and can be evenly or unevenly spaced apart from one another. In one embodiment, frozen zones 113 can be in a range about 1 μm to about 5 mm. The shape, depth, and configuration of the unfrozen zones 115 can be selected based on tissue characteristics and procedure to be performed.

The unfrozen zone 115 can be a continuous or discontinuous area between the frozen zones 113. The pattern, sizes of protected regions, locations, density, and/or a rate of creation of the zones, whether frozen zones 113 or unfrozen zones 115. The frozen zone(s) 113 will be devoid of the freeze depressant and therefore will likely freeze and experience freeze injuries. The applicator can have a generally polygonal shape (e.g., rounded square or rectangular shape) to cool a polygonal area 117, illustrated in FIG. 1B with a square-shaped pattern of frozen zones 113 (one identified). The configurations of the applicator, freeze depressant release structure, and fractional patterns be selected based on desired characteristics of the zone pattern (e.g., patterns with uniform or variable spacing, regular patterns, irregular patterns, etc.), such as number of treatment zones, density of treatment zones, etc. Applicators, freeze depressant release structures, and fractional patterns are discussed in connection with FIGS. 1A-8B.

Referring again to FIG. 1, the applicator 103 can be a vacuum applicator suitable for being applied at the back region, and the belt applicator can be applied around the thigh region, either with or without massage or vibration. Exemplary applicators and their configurations usable or adaptable for use with the treatment system 100 variously are described in, for example, commonly assigned U.S. Pat. No. 7,854,754 and U.S. Patent Publication Nos. 2008/0077201, 2008/0077211 and 2008/0287839. In further embodiments, the system 100 may also include a patient protection device (not shown) incorporated into or configured for use with the applicator 103 that prevents the applicator from directly contacting a patient's skin and thereby reducing the likelihood of cross-contamination between patients, minimizing cleaning requirements for the applicator. The patient protection device may also include or incorporate various storage, computing, and communications devices, such as a radio frequency identification (RFID) component, allowing for example, use to be monitored and/or metered. Exemplary patient protection devices are described in commonly assigned U.S. Patent Publication No. 2008/0077201.

The system 100 can also include the treatment unit 106 and supply and return fluid lines 104 between the applicator 103 and the treatment unit 106. A treatment unit 106 is a device that can increase or decrease the temperature at a connected applicator 103 that is configured to engage the subject and/or the target region of the subject. The treatment unit 106 can remove heat from a circulating coolant to a heat sink and provide a chilled coolant to the applicator 103 via the fluid lines 104. Alternatively, the treatment unit 106 can circulate warm coolant to the applicator 103 during periods of warming. In further embodiments, the treatment unit 106 can circulate coolant through the applicator 103 and increase or decrease the temperature of the applicator by controlling power delivery to one or more Peltier-type thermoelectric elements incorporated within the applicator. Examples of the circulating coolant include water, glycol, synthetic heat transfer fluid, oil, a refrigerant, and/or any other suitable heat conducting fluid. The fluid lines 104 can be hoses or other conduits constructed from polyethylene, polyvinyl chloride, polyurethane, and/or other materials that can accommodate the particular circulating coolant. The treatment unit 106 can be a refrigeration unit, a cooling tower, a thermoelectric chiller, or any other device capable of removing heat from a coolant. In one embodiment, the treatment unit 106 can include a fluid housing 105 configured to house and provide the coolant. Alternatively, a municipal water supply (e.g., tap water) can be used in place of or in conjunction with the treatment unit 106. In a further embodiment, the applicator 103 can be a fluid-cooled applicator capable of achieving a desired temperature profile such as those described in U.S. patent application Ser. No. 13/830,027, incorporated herein by reference in its entirety. One skilled in the art will recognize that there are a number of other cooling technologies that could be used such that the treatment unit, chiller, and/or applicator need not be limited to those described herein.

In the illustrated example, the applicator 103 is associated with at least one treatment unit 106. The applicator 103 can provide mechanical energy to create a vibratory, massage, and/or pulsatile effect. The applicator 103 can include one or more actuators, such as, motors with eccentric weight, or other vibratory motors such as hydraulic motors, electric motors, pneumatic motors, solenoids, other mechanical motors, piezoelectric shakers, and so on, to provide vibratory energy or other mechanical energy to the treatment site. Further examples include a plurality of actuators for use in connection with a single applicator 103 in any desired combination. For example, an eccentric weight actuator can be associated with one section of an applicator 103, while a pneumatic motor can be associated with another section of the same applicator 103. This, for example, would give the operator of the treatment system 100 options for differential treatment of lipid-rich cells within a single region or among multiple regions of the subject 101. The use of one or more actuators and actuator types in various combinations and configurations with an applicator 103 may be possible.

The applicator 103 can include one or more heat-exchanging units. Each heat-exchanging unit can include or be associated with one or more Peltier-type thermoelectric elements, and the applicator 103 can have multiple individually controlled heat-exchanging zones (e.g., between 1 and 50, between 10 and 45; between 15 and 21, approximately 100, etc.) to create a custom spatial cooling profile and/or a time-varying cooling profile. Each custom treatment profile can include one or more segments, and each segment can include a specified duration, a target temperature, and control parameters for features such as vibration, massage, vacuum, and other treatment modes. Applicators having multiple individually controlled heat-exchanging units are described in commonly assigned U.S. Patent Publication Nos. 2008/0077211 and 2011/0238051.

The control module 106 can include a fluid system 105 (illustrated in phantom line), a power supply 110 (illustrated in phantom line), and a controller 114 carried by a housing 127 with wheels 129. The fluid system 105 can include a fluid chamber and a refrigeration unit, a cooling tower, a thermoelectric chiller, heaters, or any other device capable of controlling the temperature of coolant in the fluid chamber. The coolant can be continuously or intermittently delivered to the applicator 103 via a supply fluid line and can circulate through the applicator 103 to absorb heat. The coolant, which has absorbed heat, can flow from the applicator 103 back to the control module 106 via a return fluid line. For warming periods, the control module 106 can heat the coolant that is circulated through the applicator 103. Alternatively, a municipal water supply (e.g., tap water) can be used in place of or in conjunction with the control module 106.

A pressurization device 123 can provide suction to the applicator 103 via a vacuum line and can include one or more vacuum sources (e.g., pumps). Air pockets between the subject's tissue can impair heat transfer with the tissue and, if large enough, can affect treatment. The pressurization device 123 can provide a sufficient vacuum to eliminate air gaps such that substantially no air gaps impair non-invasively cooling of the subject's subcutaneous lipid-rich cells to a treatment temperature. When the air pockets are eliminated, the tissue can be suitably cooled even though the air-egress features may contain small volumes of air and may not contact the subject's skin.

Air pressure can be controlled by a regulator located between the pressurization device 123 and the applicator 103. The control module 106 can control the vacuum level to, for example, install the liner assembly and/or draw tissue into the applicator 103 while maintaining a desired level of comfort. If the vacuum level is too low, a liner assembly, tissue, etc. may not be drawn adequately (or at all) into and/or held within the applicator 103. If the vacuum level is too high when preparing the applicator, a liner assembly can break (e.g., rupture, tear, etc.). If the vacuum level is too high during treatment, the patient can experience discomfort, bruising, or other complications. According to certain embodiments, approximately 0.5 inch Hg, 1 inch Hg, 2 inches Hg, 3 inches Hg, 5 inches Hg, 7 inches Hg, 8 inches Hg, 10 inches Hg, or 12 inches Hg vacuum is applied to draw or hold the liner assembly, tissue, etc. In some embodiments, air-egress features 180 can be configured to maintain the airflow paths when 12 inches Hg vacuum level is used to draw tissue into the tissue-receiving cavity. The number, dimensions, and positions of the air-egress features 180 can be selected to achieve desired tissue contact. Other vacuum levels can be selected based on the characteristics of the tissue, desired level of comfort, and vacuum leakage rates. Vacuum leak rates of the applicator 103 can be equal to or less than about 0.2 LPM, 0.5 LPM, 1 LPM, or 2 LPM at the pressure levels disclosed herein. For example, the vacuum leak rate can be equal to or less than about 0.2 LPM at 8 inches Hg, 0.5 LPM at 8 inches Hg, 1 LPM at 8 inches Hg, or 2 LPM at 8 inches Hg. The configuration of the pressurization device 123 and applicator 103 can be selected based on the desired vacuum levels, leakage rates, and other operating parameters.

The power supply 110 can provide a direct current voltage for powering electrical elements of the applicator 103 via a line. The electrical elements can be thermal devices, sensors, actuators, controllers (e.g., a controller integrated into the applicator 103), or the like. An operator can use an input/output device in the form of a screen 118 (“input/output device 118”) of the controller 114 to control operation of the treatment system 100, and the input/output device 118 can display the state of operation of the treatment system 100 and/or progress of a treatment protocol. In some embodiments, the controller 114 can exchange data with the applicator 103 via the line, a wireless communication link, or an optical communication link and can monitor and adjust treatment based on, without limitation, one or more treatment profiles and/or patient-specific treatment plans, such as those described, for example, in commonly assigned U.S. Pat. No. 8,275,442. The controller 114 can contain instructions to perform the treatment profiles and/or patient-specific treatment plans, which can include one or more segments, and each segment can include temperature profiles, vacuum levels, and/or specified durations (e.g., 1 minute, 5 minutes, 10 minutes, 20 minutes, 30 minutes, 1 hour, 2 hours, etc.). For example, the controller 114 can be programmed to cause the pressurization device to operate to pull tissue into the applicator. After tissue draw, the pressurization device can operate to hold the subject's skin in thermal contact appropriate features while the cup conductively cools tissue. If the sensor detects tissue moving out of thermal contact with the cup, the vacuum can be increased to reestablish suitable thermal contact. In some embodiments, the controller 114 is programmed to cause the pressurization device to provide a sufficient vacuum to keep substantially all of each region of the temperature-controlled surface between air-egress features in thermal contact with the subject's skin. This provides a relatively large contact interface for efficient heat transfer with the target tissue.

Different vacuum levels can be utilized during treatment sessions. For example, relatively strong vacuums can be used to pull the subject's tissue into the applicator. A weaker vacuum can be maintained to hold the subject's tissue against the thermally conductive surface. If suitable thermal contact is not maintained (e.g., the subject's skin moves away from the thermally conductive surface), the vacuum level can be increased to reestablish suitable thermal contact. In other procedures, a generally constant vacuum level can be used throughout the treatment session.

If the treatment system 100 includes multiple applicators, a treatment profile can include specific profiles for each applicator to concurrently or sequentially treat multiple treatment sites, including, but not limited to, sites along the subject's torso, abdomen, legs, buttock, legs, face and/or neck (e.g., submental sites, submandibular sites, etc.), knees, back, arms, ankle region, or other treatment sites. The vacuum levels can be selected based on the configuration of the cup. Strong vacuum levels can be used with relatively deep cups whereas weak vacuum levels can be used with relatively shallow cups. The vacuum level and cup configuration can be selected based on the treatment site and desired volume of tissue to be treated. In some embodiments, the controller 114 can be incorporated into the applicator 103 or another component of the treatment system 100.

The system 100 can further include a power supply 110 and a controller 114 operatively coupled to the applicator 103. In one embodiment, the power supply 110 can provide a direct current voltage to the applicator 103 to remove heat from the subject 101. The controller 114 can monitor process parameters via sensors (not shown) placed proximate to the applicator 103 via a control line 116 to, among other things, adjust the heat removal rate and/or energy delivery rate based on the process parameters. The controller 114 can further monitor process parameters to adjust the applicator 103 based on treatment parameters, such as treatment parameters defined in a custom treatment profile or patient-specific treatment plan, such as those described, for example, in commonly assigned U.S. Pat. No. 8,275,442.

The controller 114 can exchange data with the applicator 103 via an electrical line or, alternatively, via a wireless or an optical communication link. The control line and electrical line can either lack any support structure or may be bundled into or otherwise accompanied by a conduit or the like to protect such lines, enhance ergonomic comfort, minimize unwanted motion (and thus potential inefficient removal of heat from and/or delivery of energy to subject 101), and to provide an aesthetic appearance to the system 100. Examples of such a conduit include a flexible polymeric, fabric, or composite sheath, an adjustable arm, etc. Such a conduit (not shown) may be designed (via adjustable joints, etc.) to “set” the conduit in place for the treatment of the subject 101.

The controller 114 can include any processor, Programmable Logic Controller, Distributed Control System, secure processor, and the like. A secure processor can be implemented as an integrated circuit with access-controlled physical interfaces; tamper resistant containment; means of detecting and responding to physical tampering; secure storage; and shielded execution of computer-executable instructions. Some secure processors also provide cryptographic accelerator circuitry. Secure storage may also be implemented as a secure flash memory, secure serial EEPROM, secure field programmable gate array, or secure application-specific integrated circuit.

In another embodiment, the controller 114 can receive data from an input device 118 (shown as a touch screen), transmit data to an output device, and/or exchange data with a control panel (not shown). The input device 118 can include a keyboard, a mouse, a stylus, a touch screen, a push button, a switch, a potentiometer, a scanner, an audio component such as a microphone, or any other device suitable for accepting user input. The output device can include a display or touch screen, a printer, a video monitor, a medium reader, an audio device such as a speaker, any combination thereof, and any other device or devices suitable for providing user feedback.

In the embodiment of FIG. 1, the output device is a touch screen that functions as both an input device 118 and an output device. The control panel can include visual indicator devices or controls (e.g., indicator lights, numerical displays, etc.) and/or audio indicator devices or controls. The control panel may be a component separate from the input device 118 and/or output device, may be integrated with one or more of the devices, may be partially integrated with one or more of the devices, may be in another location, and so on. In alternative examples, the control panel, input device 118, output device, or parts thereof (described herein) may be contained in, attached to, or integrated with the applicator 103. In this example, the controller 114, power supply 110, control panel, treatment unit 106, input device 118, and output device are carried by a housing 127 with wheels 129 for portability. In alternative embodiments, the controller 114 can be contained in, attached to, or integrated with the multi-modality applicator 103 and/or the patient protection device described above. In yet other embodiments, the various components can be fixedly installed at a treatment site. Further details with respect to components and/or operation of applicators 103, treatment units 106, and other components may be found in commonly assigned U.S. Patent Publication No. 2008/0287839.

In operation, and upon receiving input to start a treatment protocol, the controller 114 can cause one or more power supplies 110, one or more treatment units 106, and one or more applicators 103 to cycle through each segment of a prescribed treatment plan. In so doing, power supply 110 and treatment unit 106 provide coolant and power to one or more functional components of the applicator 103, such as thermoelectric coolers (e.g., TEC “zones”), to begin a cooling cycle and, for example, activate features or modes such as vibration, massage, vacuum, etc.

Using temperature sensors (not shown) proximate to the one or more applicators 103, the patient's skin, a patient protection device, or other locations or combinations thereof, the controller 114 can determine whether a temperature or heat flux is sufficiently close to the target temperature or heat flux. It will be appreciated that while a region of the body (e.g., adipose tissue) has been cooled or heated to the target temperature, in actuality that region of the body may be close but not equal to the target temperature (e.g., because of the body's natural heating and cooling variations). Thus, although the system may attempt to heat or cool the tissue to the target temperature or to provide a target heat flux, a sensor may measure a sufficiently close temperature or heat flux. If the target temperature has not been reached, power can be increased or decreased to change heat flux to maintain the target temperature or “set-point” selectively to affect lipid-rich subcutaneous adipose tissue.

When the prescribed segment duration expires, the controller 114 may apply the temperature and duration indicated in the next treatment profile segment. In some embodiments, temperature can be controlled using a variable other than or in addition to power.

In some embodiments, heat flux measurements can indicate other changes or anomalies that can occur during treatment administration. For example, an increase in temperature detected by a heat flux sensor can indicate a freezing event at the skin or underlying tissue (i.e., dermal tissue). An increase in temperature as detected by the heat flux sensors can also indicate movement associated with the applicator, causing the applicator to contact a warmer area of the skin, for example. Methods and systems for collection of feedback data and monitoring of temperature measurements are described in commonly assigned U.S. Pat. No. 8,285,390.

The applicators 103 may also include additional sensors to detect process treatment feedback. Additional sensors may be included for measuring tissue impedance, treatment application force, tissue contact with the applicator, and energy interaction with the skin of the subject 101 among other process parameters.

In one embodiment, feedback data associated heat removal from lipid-rich cells in the cutaneous or subcutaneous layer can be collected in real-time. Real-time collection and processing of such feedback data can be used in concert with treatment administration to ensure that the process parameters used to alter or reduce subcutaneous adipose tissue are administered correctly and efficaciously.

Examples of the system 100 may provide the applicator 103 which damages, injures, disrupts, or otherwise reduces frozen cells and/or structures generally without collateral damage to unfrozen cells and/or structures in the treatment region. In general, it is believed that lipid-rich cells selectively can be affected (e.g., damaged, injured, or disrupted) by exposing such cells to low temperatures that do not so affect non-lipid-rich cells. Moreover, as discussed above, a freeze depressant can be administered topically to the skin of the subject 101 at the site and/or used with the applicator 103 to, among other advantages, assist in preventing freezing of the non-lipid-rich tissue (e.g., in the dermal and epidermal skin layers) during treatment to selectively interrogate lipid-rich cells in the treatment region so as to beneficially and cosmetically alter subcutaneous adipose tissue, treat sweat glands, and/or reduce sebum secretion. As a result, lipid-rich cells, such as subcutaneous adipose tissue and glandular epithelial cells, can be damaged while other non-lipid-rich cells (e.g., dermal and epidermal skin cells) in the same region are generally not damaged even though the non-lipid-rich cells at the surface may be subject to even lower temperatures. In some embodiments, the mechanical energy provided by the applicator 103 may further enhance the effect on lipid-rich cells by mechanically disrupting the affected lipid-rich cells. In one mode of operation, the applicator 103 may be configured to be a handheld device such as the device disclosed in commonly assigned U.S. Pat. No. 7,854,754.

Applying the applicator 103 with pressure or with a vacuum type force to the subject's skin or pressing against the skin can be advantageous to achieve efficient treatment. In general, the subject 101 has an internal body temperature of about 37° C., and the blood circulation is one mechanism for maintaining a constant body temperature. As a result, blood flow through the skin and subcutaneous layer of the region to be treated can be viewed as a heat source that counteracts the cooling of the subdermal fat. As such, cooling the tissue of interest requires not only removing the heat from such tissue but also that of the blood circulating through this tissue. Thus, temporarily reducing or eliminating blood flow through the treatment region, by means such as, for example, applying the applicator with pressure, can improve the efficiency of tissue cooling and avoid excessive heat loss through the dermis and epidermis. Additionally, a vacuum can pull skin away from the body which can assist in cooling targeted underlying tissue.

### D. Suitable Applicators

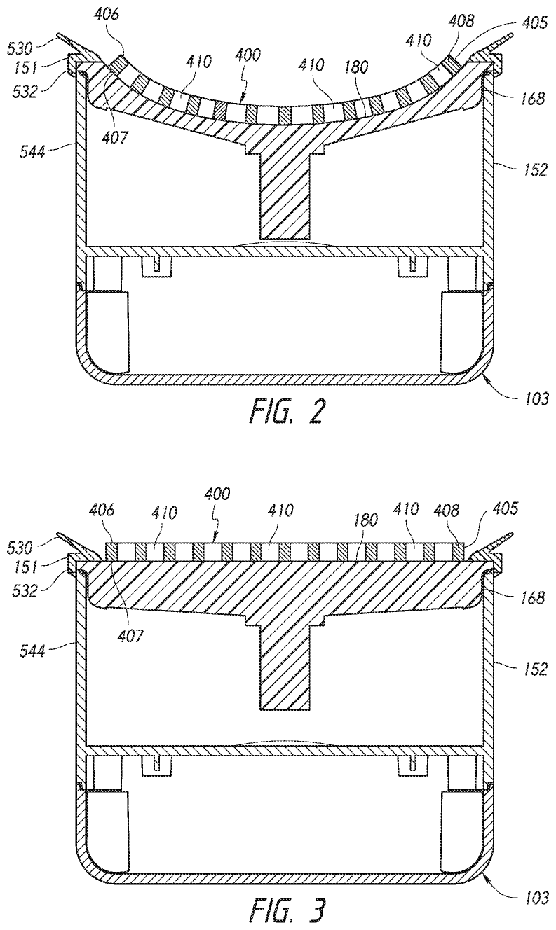

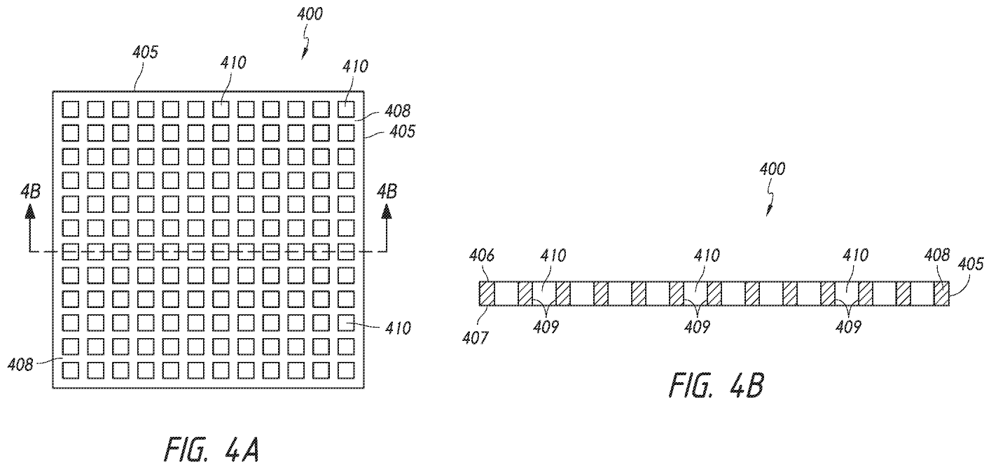

FIG. 2 is a cross-sectional view of the applicator 103 for non-invasively removing heat from a region and/or otherwise altering one or more target structures in the subject's dermis and/or hypodermis of the subject taken along line 2-2 of FIG. 1. The applicator 103 includes a contoured sealing element 151 and a base unit 152. The sealing element 151 can conform closely to contours of the subject's body to sealingly engage a freeze depressant release structure 400 (discussed in greater detail with respect to FIGS. 4A and 4B) or skin surface. The base unit 152 can include a cup 168 for holding tissue. If the freeze depressant release structure 400 is used with the applicator 103, the release structure 400 can line the cup 168 and can be perforated such that a vacuum can be drawn (e.g., via vacuum lines 401) to urge the subject's skin against the freeze depressant release structure 400, thereby maintaining thermal contact between the tissue and the cup 168.

The sealing element 151 can include a contoured lip 530 and a body 532. The lip 530 can be defined as an entrance and can be configured to sealingly engage, for example, the subject's skin. For example, the lip 530 can be configured for forming airtight seals with the subject's skin and can be made, in whole or in part, of silicon, rubber, soft plastic, or other suitable highly compliant materials. The mechanical properties, thermal properties, shape, and/or dimensions of the contoured lip 530 can be selected based on, for example, whether it contacts the subject's skin, liner assembly, a cryoprotectant gel pad, or the like. The body 532 is coupled to a housing 544. As tissue is pulled through the entrance of the sealing element 151 and toward the cup 168, the contoured lip 530 can deflect outwardly.

Different sealing elements or cups 168 can be installed on the base unit 152 for treatment flexibility. The geometries of the contoured sealing element 151 and cup 168, which can be replaced, can be selected to conform to a contour of a cutaneous layer. The sides, waistline, and other features of the contoured heads can be selected to facilitate conformation of heads to the contours of individual target areas. For example, the shape of a typical human torso may vary between having a relatively large radius of curvature (e.g., on the stomach or back) and having a relatively small radius of curvature (e.g., on the abdominal sides). Moreover, the size of a cup having an approximately consistent curvature may vary or may be generally planar (FIG. 3). The sealing elements may be fitted to individual lipid-rich cell deposits to achieve an approximately air-tight seal, achieve the vacuum pressure for drawing tissue into an interior cavity for treatment, maintain suction to hold the tissue, massage tissue (e.g., by altering pressure levels), and use little or no force to maintain contact between an applicator and a patient.

Components can be attached and detached in a plurality of combinations to achieve a desired contour for a treatment. Accordingly, a single base unit and/or umbilical cable may be combined with a set of interchangeable heads and/or cups to form a wide variety of contours for treating different lipid-rich cell deposits in a cost-effective manner. Further, a practitioner performing the treatment can demonstrate their expertise to the patient by tailoring the applicator contour to the specific body parts being treated. In this manner, the patient understands that their treatment is customized to their body for better comfort and for better treatment results.

Tissue-receiving cavities and cups 168 disclosed herein can have substantially U-shaped cross sections, V-shaped cross sections, or partially circular/elliptical cross-sections, as well as or other cross sections suitable for receiving tissue. Thus, thermal properties, shape, and/or configuration of the cup 168 can be selected based on, for example, target treatment temperatures and/or volume of the targeted tissue. Embodiments of the base units for treating large volumes of tissue (e.g., adipose tissue along the abdomen, hips, buttock, etc.) can have a maximum depth equal to or less than about 2 cm, 5 cm, 10 cm, 15 cm, 20 cm, or 30 cm, for example. Embodiments of the base units for treating small volumes (e.g., a small volume of submental tissue) can have a maximum depth equal to or less than about 0.5 cm, 2 cm, 2.5 cm, 3 cm, or 5 cm, for example. The maximum depth of the tissue-receiving cavity can be selected based on, for example, the volume of targeted tissue, characteristics of the targeted tissue, and/or desired level of patient comfort.

In some embodiments, the applicator 103 can include one or more air-egress features 180 (e.g., vacuum ports) that can help distribute a vacuum across the cup/tissue interface to enhance patient comfort and prevent air gaps at the tissue/cup interface during tissue draw. Each air-egress feature 180 can be positioned within one of the channels to pull tissue against the applicator 103.

Referring again to FIG. 1, the control module 106 can include a fluid system 105 (illustrated in phantom line), a power supply 110 (illustrated in phantom line), and a controller 114 carried by a housing 127 with wheels 129. The fluid system 105 can include a fluid chamber and a refrigeration unit, a cooling tower, a thermoelectric chiller, heaters, or any other device capable of controlling the temperature of coolant in the fluid chamber. The coolant can be continuously or intermittently delivered to the applicator 103 via a supply fluid line and can circulate through the applicator 103 to absorb heat. The coolant, which has absorbed heat, can flow from the applicator 103 back to the control module 106 via a return fluid line. For warming periods, the control module 106 can heat the coolant that is circulated through the applicator 103. Alternatively, a municipal water supply (e.g., tap water) can be used in place of or in conjunction with the control module 106.

The power supply 110 can provide a direct current voltage for powering electrical elements of the applicator 103 via a line. The electrical elements can be thermal devices, sensors, actuators, controllers (e.g., a controller integrated into the applicator 103), or the like. An operator can use an input/output device in the form of a screen 118 (“input/output device 118”) of the controller 114 to control operation of the treatment system 100, and the input/output device 118 can display the state of operation of the treatment system 100 and/or progress of a treatment protocol. In some embodiments, the controller 114 can exchange data with the applicator 103 via the line, a wireless communication link, or an optical communication link and can monitor and adjust treatment based on, without limitation, one or more treatment profiles and/or patient-specific treatment plans, such as those described, for example, in commonly assigned U.S. Pat. No. 8,275,442. The controller 114 can contain instructions to perform the treatment profiles and/or patient-specific treatment plans, which can include one or more segments, and each segment can include temperature profiles, vacuum levels, and/or specified durations (e.g., 1 minute, 5 minutes, 10 minutes, 20 minutes, 30 minutes, 1 hour, 2 hours, etc.). For example, the controller 114 can be programmed to cause the pressurization device to operate to pull tissue into the applicator. After drawing the tissue, the pressurization device can hold the subject's skin in thermal contact appropriate features while the cup conductively cools tissue. If the sensor detects tissue moving out of thermal contact with the cup, the vacuum can be increased to reestablish suitable thermal contact.

If the treatment system 100 includes multiple applicators, a treatment profile can include specific profiles for each applicator to concurrently or sequentially treat multiple treatment sites, including, but not limited to, sites along the subject's torso, abdomen, legs, buttock, legs, face and/or neck (e.g., submental sites, submandibular sites, etc.), knees, back, arms, ankle region, or other treatment sites. The vacuum levels can be selected based on the configuration of the cup. Strong vacuum levels can be used with relatively deep cups whereas weak vacuum levels can be used with relatively shallow cups. The vacuum level and cup configuration can be selected based on the treatment site and desired volume of tissue to be treated. In some embodiments, the controller 114 can be incorporated into the applicator 103 or another component of the treatment system 100.

Some embodiments according to the present technology may use a cryoprotectant including a freezing point depressant that can assist in preventing freezing of non-lipid-rich tissue (e.g., dermal and epidermal tissue) during treatment. Suitable freeze depressants and processes for implementing freeze depressants are described herein and in commonly assigned U.S. Patent Publication No. 2007/0255362. The cryoprotectant can be part of a freeze depressant composition that may additionally include one or more anti-freeze proteins, a thickening agent, a pH buffer, a humectant, a surfactant, and/or other additives as described herein. The cryoprotectant depressant may include, for example, propylene glycol (PG), polyethylene glycol (PEG), dimethyl sulfoxide (DMSO), or other suitable alcohol compounds. In a particular embodiment, a freezing point depressant may include about 40% propylene glycol and about 60% water. In other embodiments, a freeze depressant may include about 30% propylene glycol, about 30% glycerin (a humectant), and about 40% ethanol. In another embodiment, a freeze depressant may include about 40% propylene glycol, about 0.8% hydroxyethyl cellulose (a thickening agent), and about 59.2% water. In a further embodiment, a freeze depressant may include about 50% polypropylene glycol, about 40% glycerin, and about 10% ethanol. In yet a further embodiment, the freeze depressant can include about 30-50% by volume of one or more freezing point depressants and include about 50% wt./vol. to about 70% wt./vol. of a combination of one or more of a thickening agent, a pH buffer, a humectant, a surfactant, one more additives and/or one or more anti-freeze proteins.

### E. Structures for Release of Cryoprotectant

As discussed above under Headings A-D, systems of the present technology include sustained and/or replenishing release of freeze depressant compositions (e.g., cryoprotectant) can be provided by the freeze depressant release structure (400 of FIGS. 1A-3). Several embodiments of the system can include structures for enhancing sustained and/or replenishing release of freeze depressant to a treatment site. For example, FIG. 4A is a partially schematic, isometric view illustrating a freeze depressant release structure suitable to be used in the treatment system of FIG. 1 (the freeze depressant release structure 400 of FIGS. 2 and 3) in accordance with an embodiment of the present technology. The freeze depressant release structure 400 can be configured to absorb and/or otherwise hold a freezing point depressant composition (e.g., cryoprotectant) and release the freeze depressant in a time dependent manner to the subject's skin and/or the applicator 103 (FIGS. 1-3). Accordingly, the release structure 400 can be configured to be placed on the subject's skin at the site prior to the placement of the applicator 103. In another embodiment, the release structure 400 can be adhered to the applicator 103 such that it comes in contact with the subject's skin as the applicator 103 is positioned at the site.

The release structure 400 includes sidewalls 405 having one or more cross-sectional dimensions that can be selected based on, but not limited to, one or more of the following factors: duration of treatment, temperatures of the treatment, type of freeze depressant, type of applicator, formulation of the freeze depressant and its physical properties (e.g., viscosity, surface tension, etc.) and others. In some embodiments, the release structure 400 is formed of a porous material 408 which can be any number of porous materials having a variety of pore sizes and/or densities. The material can be selected based on factors generally similar to those considered when selecting the cross-sectional dimension of the release structure 400 and includes, but is not limited to, cotton (e.g., Webril), paper, hydrogel, or the like.

In one embodiment, the release structure 400 can include an absorbent containing a bioabsorbable freezing point depressant (e.g., a cryoprotectant). The absorbent can be constructed from cotton material and/or gauze material and the freezing point depressant can be absorbed on and/or therein. In some embodiments, and while the subject is being treated, the absorbent can be positioned between the subject's skin and a heat-exchanging surface of a treatment device or applicator 103. A liner or protective sleeve may be positioned between the absorbent and the applicator 103 to shield the applicator and to provide a sanitary barrier that is, in some embodiments, inexpensive and thus disposable.

In another embodiment, the release structure 400 can be a microporous or gel pad. For example, the freeze point depressant (e.g., cryoprotectant) can be absorbed or delivered within the microporous or gel pad that is positioned between the subject's skin and a heat-exchanging surface of a treatment device or applicator 103. The gel pad can release the cryoprotectant to the subject's skin either prior to or during treatment. In some embodiments, the microporous gel pad can continually release quantities of freeze point depressant over time and/or during a treatment session. In some embodiments, the freeze point depressant can be released at higher concentrations, higher volumes and/or at more controlled rates than by conventional spreading of a cryoprotectant on the skin of the subject.

In some embodiments, the release structure 400 can be configured to continuously or periodically release the freeze depressant. The release rate of freeze depressant can be related to the absorption and/or dispersion rate of the freeze depressant and/or one or more physical features of the release structure 400, such as the material, cross-sectional dimension of the sidewalls 405, amongst others. In one embodiment, the release structure 400 can deliver freeze depressant at a generally constant rate throughout most or all of the treatment process. In other embodiments, the release structure 400 can maintain contact between the subject's skin and the freeze depressant. The subject's skin can absorb the freeze depressant to inhibit freezing, prevent, or limit freeze damage to unfrozen tissue. The freeze depressant can maintain unfrozen zones having unfrozen cells and sharp, needle-like ice crystals surrounding but not damaging or otherwise destroying the cells.

The release structure 400, in some embodiments, can continuously deliver freeze depressant for a duration of time equal to or greater than about 5 minutes, about 10 minutes, about 15 minutes, about 20 minutes, about 30 minutes, 1 hour, or 2 hours. The release structure 400 can be replaced for longer treatments. In other embodiments, the release structure 400 can be configured to be reloadable during treatment so that, for example, the release structure 400 can continue to deliver cryoprotectant for a longer duration of time.

In some embodiments, the release structure 400 can include an adhesive (e.g., tape strips, textile tapes, etc.), such that the release structure 400 can be releasably retained on the surface of the skin at the treatment site. Accordingly, in such embodiments, the freeze depressant is retained or sealed against the surface of the skin at the site.

The release structure 400 includes an array of channels 410 arranged into a grid and extending through the release structure 400 from a first side 406 to a second side 407. While not intending to be limiting, the channels 410 can be formed from a uniform carrier material by punching or etching (e.g., chemical, laser, etc.) and include sidewalls 409. Channels 410 are also illustrated in FIG. 4B, which is a schematic, cross-sectional view of the release structure 400 taken along line 4B of FIG. 4A. Each channel 410 extends through the release structure 400 allowing direct contact between the subject's skin and the cup 168 of FIGS. 2 and 3 (e.g., the freeze depressant is not delivered to the site through any of the channels 410) to freeze the subject's epidermis. The freeze depressant is carried within the porous material 408 to the subject's epidermal layer thereby protecting the epidermis from freezing at areas 408. In some embodiments, use of the release structure channels 410 to freeze the epidermis while delivering the freeze depressant to the epidermis in areas 408 to prevent freezing that can cause fractional freezing of the site. While not intending to be bound by any particular theory, the release structure 400 provides a generally continuous freeze through each channel 410 to the subject's epidermis thereby resulting in a faster, more efficient treatment compared to fractional freezing treatments using discontinuous applicators which deliver a reduced cooling load compared to those of the present technology.

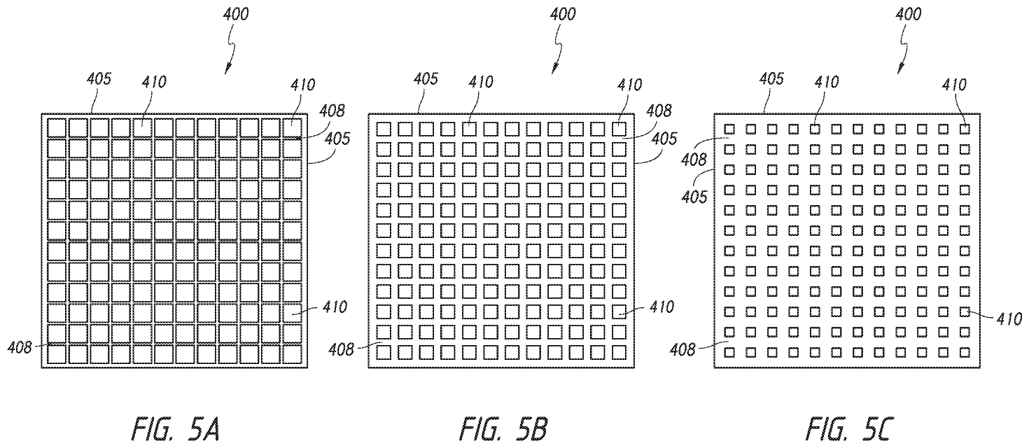

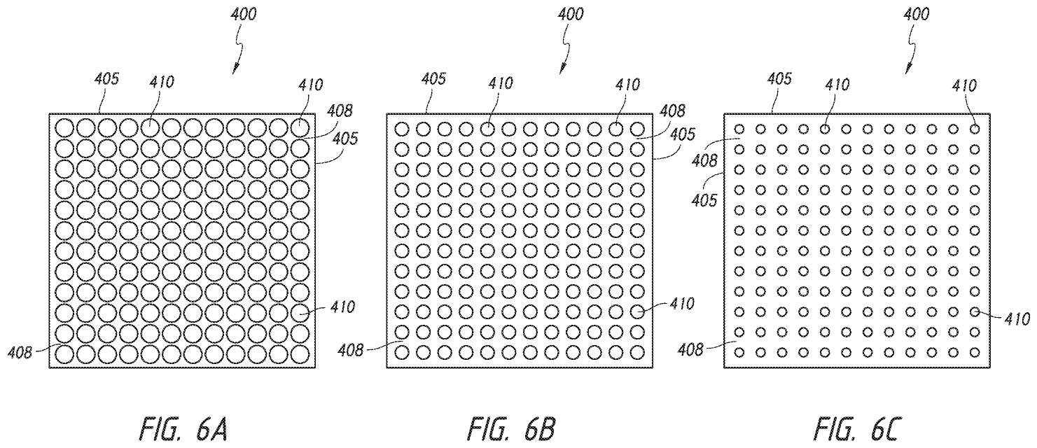

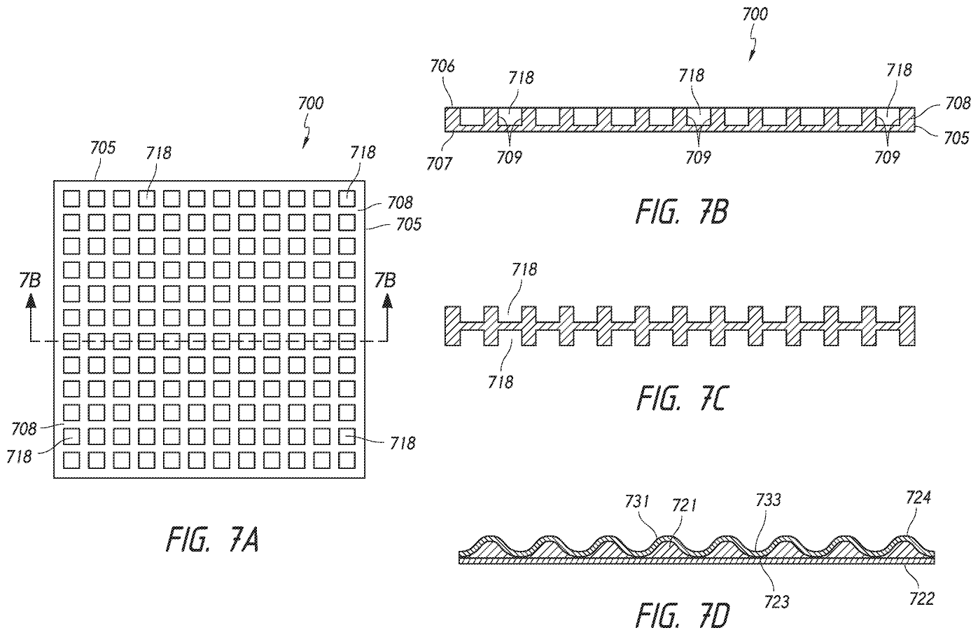

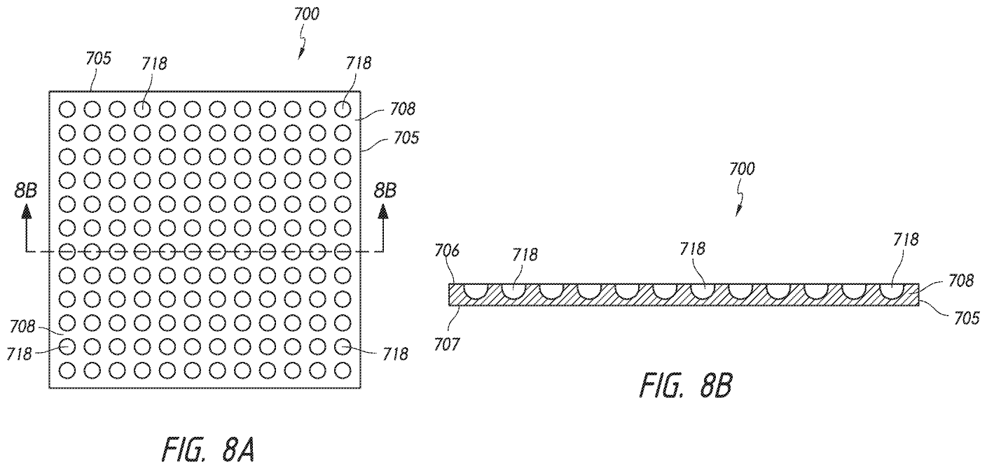

As shown in FIGS. 5A-6C, the release structure 400 can include channels 410 having cross-sectional sizes, cross-sectional shapes, and spacings apart which differ from those illustrated in FIG. 4A. In some embodiments, the channels 410 can have similar cross-sectional shapes to those illustrated in FIG. 4A while the cross-sectional dimensions can be larger (FIG. 5A), generally similar (FIG. 5B) or smaller (FIG. 5C) than those illustrated in FIG. 4A. In other embodiments, the cross-sectional shapes of the channels 410 can be generally circular (FIGS. 6A-6C) compared to the generally cross-sectional square shapes of the channels 410 illustrated in FIG. 4A. Similar to FIGS. 5A-5C, the cross-sectional circular channels 410 of FIGS. 6A-6C can have cross-sectional dimensions that are larger (FIG. 6A), generally similar (FIG. 6B) or smaller (FIG. 6C) than those illustrated in FIG. 4A. As the cross-sectional dimensions of the channels 410 increase (FIGS. 5A and 6A) the spacing of each channel 410 apart from other channels 410 decreases whereas as the cross-sectional dimensions of the channels 410 decrease (FIGS. 5C and 6C) the spacing of each channel 410 apart from other channels 410 increases. The size, shape, and spacings of the channels 410 in the release structure 400 can be selected based on certain treatment parameters, such as duration, temperature, cycles, and the like.

In other embodiments, a release structure 700 having sidewalls 705, a first side 706, and a second side 707 includes one or more chambers 718 (FIG. 7A) rather than channels 410 (FIG. 4). The chambers 718 are shown in FIG. 7B, which is a partially schematic, isometric view illustrating a cross-section of the freeze depressant release structure of FIG. 7A taken along line 7B in accordance with yet another embodiment of the technology. Unlike the channels 410, chambers 718 include an opening at a first side 706 of the release structure 700 and extend toward, but not entirely through, the second side 707. Rather, the porous material 708 extends across a continuous length of the second side 707 of the release structure 700 and into sidewalls 709, forming the chambers 718 which are devoid of cryoprotectant. FIG. 7C shows another embodiment where the chambers extend from both sides 706, 707 of the release structure such that the release structure can optionally have a symmetrical configuration. The release structure 700 can be positioned on a patient such that the patient skin 724 (FIG. 7D) is in contact with either the first or second side 706, 707, with the applicator being positioned on an opposite side of the release structure. Referring to FIG. 7D, in use, since the release structure is made of compliant material, and since the patient skin or tissue in contact with the releases structure is also compliant, the voids 718 existent in the release structure prior to treatment will be flattened out and will disappear due to compressive forces, such that in use thicker sections 721 of release structure will exist between the skin 731 and the applicator 722 in areas laterally adjacent where the voids were prior to use, and thinner sections 723 of release structure will exist between the skin 733 and the applicator in areas where the voids 718 previously existed. In these embodiments, since the sections 721 are thicker than the sections 723, thermal heat transfer through the sections 721 will be less than thermal heat transfer through the sections 723, which will cause the skin 733 adjacent to sections 723 to cool faster than the skin 731 adjacent sections 721. Additionally, since the sections 721 are thicker than the sections 723, more cryoprotectant from the release structure will be transferred to the skin 731 as compared to that transferred to the skin 733. Hence, these two effects will cause the skin 733 to preferentially freeze, or freeze faster, than the skin 731 Unlike the release structure 400 having channels 410, chambers 718 may allow the epidermis to freeze at locations of the skin 733 but not at locations of the skin 731 which could facilitate formation of freezing microchannels between the epidermis and dermis in a discontinuous manner. Similar to the release structure 400 having channels 410, chambers 718 can have a variety of shapes, sizes, and spacings. For example, as shown in FIGS. 8A and 8B, the chambers 718 can have generally circular openings at the first side 706 of the release structure 700 and generally curved bases proximate to the second side 707 of the release structure 700.

While not intending to be limiting, the chambers 718 can be formed from a uniform carrier material by printing (e.g., imprinting) or etching (e.g., chemical, laser, etc.) and may be a more rapid and simpler release structure 700 to manufacture than release structure 400 or other cryoprotectant release structures (e.g., gel pads).

Although a noninvasive applicator unit is illustrated and discussed with respect to FIGS. 1-7D, minimally invasive applicators may also be employed. In such a case, the applicator and patient protection device may be integrated. As an example, a cryoprobe and/or electrode that may be inserted directly into the subcutaneous adipose tissue to cool or freeze the tissue is an example of such a minimally invasive applicator. Cryoprobes manufactured by, for example, Endocare, Inc., of Irvine, Calif. are suitable for such applications. This patent application incorporates by reference U.S. Pat. No. 6,494,844, entitled “DEVICE FOR BIOPSY AND TREATMENT OF BREAST TUMORS”; U.S. Pat. No. 6,551,255, entitled “DEVICE FOR BIOPSY OF TUMORS”; U.S. Publication No. 2007/0055173, entitled “ROTATIONAL CORE BIOPSY DEVICE WITH LIQUID CRYOGEN ADHESION PROBE”; U.S. Pat. No. 6,789,545, entitled “METHOD AND SYSTEM FOR CRYOABLATING FIBROADENOMAS”; U.S. Publication No. 2004/0215294, entitled “CRYOTHERAPY PROBE”; U.S. Pat. No. 7,083,612, entitled “CRYOTHERAPY SYSTEM”; and U.S. Publication No. 2005/0261753, entitled “METHODS AND SYSTEMS FOR CRYOGENIC COOLING”.

The treatment device or applicator, the freeze depressant, and/or other components of the treatment system 100 can be included in a kit (not shown) for removing heat from cutaneous or subcutaneous lipid-rich cells of the subject 101. The kit can also include instruction documentation containing information regarding how to (a) apply the composition to a target region and/or a heat-exchanging surface of the treatment device or applicator and (b) reduce a temperature of the target region such that lipid-rich cells in the region are affected while preserving non-lipid-rich cells proximate to the heat-exchanging surface. In other embodiments, the kit can include pre-treatment and/or post-treatment compositions. The kit can further include one or more dermatological pre-treatment and/or post-treatment components, such as a dermal agitation brush, cleaning solutions and pads, gauze, bandages, etc.

### F. Suitable Cryoprotectant Compositions

A freeze depressant suitable to be used in the treatment system 100 of FIG. 1 and/or in treatment regimens associated with use of suitable treatment systems for cooling cells, tissue, and/or structures (e.g., subcutaneous adipose tissue, glandular epithelial cells) is a substance that may protect biological tissues of a subject from freezing damage (e.g., damage due to ice formation within the tissue). The freeze depressant can be used as a pre-treatment formulation applied to the skin of the subject prior to removing heat to increase a permeability of the skin and/or to lower a freezing point of unfrozen cells, tissue, and/or structures (e.g., in epidermal and/or dermal layers). In these or other embodiments, the freeze depressant can also be used during heat removal when provided with the applicator 103 (FIG. 1) and as further described herein.

The freeze depressant may contain a freezing point depressant along with one or more other components, for example, a thickening agent, a pH buffer, a humectant, a surfactant, and/or other additives configured to provide selected properties to the compound, such as one or more anti-freeze proteins. The freeze depressant may be formulated as a non-freezing liquid (e.g., an aqueous solution or a non-aqueous solution), a non-freezing gel, a non-freezing hydrogel, or a non-freezing paste. The freeze depressant may be hygroscopic, thermally conductive, and can be biocompatible. In certain embodiments, the freeze depressant may be formulated to be acoustically transparent to allow ultrasound to pass through the freeze depressant, such as a water-based gel described in U.S. Pat. No. 4,002,221 issued to Buchalter and U.S. Pat. No. 4,459,854 issued to Richardson et al., the entire disclosures of which are incorporated herein by reference.

The freezing point depressant can include propylene glycol (PG), polyethylene glycol (PEG), polypropylene glycol (PPG), ethylene glycol, dimethyl sulfoxide (DMSO), combinations thereof, or other glycols. The freezing point depressant may also include ethanol, propanol, iso-propanol, butanol, and/or other suitable alcohol compounds. Certain freezing point depressants (e.g., PG, PPG, PEG, etc.) may also be used to improve spreadability of the freeze depressant and to provide lubrication. The freezing point depressant may lower the freezing point of a solution (e.g., body fluid) to about 0° C. to −40° C. In other embodiments the freezing point of a solution can be lowered to about −10° C. to about −20° C., about −10° C. to about −18° C., or to about −10° C. to about −15° C. In certain embodiments, the freezing point of a solution can be lowered to a temperature less than about 0° C., less than about −5° C., less than about −10° C., less than about −12° C., less than about −15° C., less than about −16° C., less than about −17° C., less than about −18° C., less than about −19° C., or less than about −20° C. For example, the freezing point depressant may lower the freezing point of a solution (e.g., body fluid) to a temperature less than about −20° C. to about −25° C., less than about −20° C. to about −30° C., less than about −25 to about −35° C., or less than about −30° C. to about −40° C.

The thickening agent can include carboxyl polyethylene polymer, hydroxyethyl xylose polymer, carboxyl methylcellulose, hydroxyethyl cellulose (HEC), and/or other viscosity modifiers to provide a viscosity in the range of about 1 cP to about 10,000 cP. In one embodiment, the thickening agent can provide a viscosity in the range of about 4,000 cP to about 8,000 cP. In another embodiment, the thickening agent can provide a viscosity in the range of about 5,000 cP to about 7,000 cP. Other viscosities can be achieved, if needed or desired. In various embodiments, a cryoprotectant having a viscosity in one or more of these ranges may readily adhere to the treatment device, the skin of the subject, and/or the interface between the treatment device and the skin of the subject during treatment.

The pH buffer may include cholamine chloride, cetamide, glycine, tricine, glycinamide, bicine, and/or other suitable pH buffers. The pH buffer may help the freeze depressant to have a consistent pH of about 3.5 to about 11.5. In other embodiments, the pH can be consistently between about 5 to about 9.5, and in further embodiments between about 6 to about 7.5. In certain embodiments, the pH of the freeze depressant may be close to the pH of the skin of the subject.

The humectant may include glycerin, alkylene glycol, polyalkylene glycol, propylene glycol, glyceryl triacetate, polyols (e.g., sorbitol and/or maltitol), polymeric polyols (e.g., polydextrose), quillaia, lactic acid, and/or urea. The humectant may promote the retention of water to prevent the freeze depressant from drying out. The surfactant may include sodium dodecyl sulfate, ammonium lauryl sulfate, sodium lauryl sulfate, alkyl benzene sulfonate, sodium lauryl ether sulfate, and other suitable surfactants. The surfactant may promote easy spreading of the freeze depressant when an operator applies the freeze depressant to the treatment device, the skin of the subject, and/or the interface between the treatment device and the skin of the subject during treatment.

In several embodiments, the freeze depressant composition also includes anti-freeze proteins that inhibit freezing of the treatment region but, that when cold enough, facilitate the controlled formation of ice crystals within target cells within the treatment region to manage thermal injury within the treatment region and surrounding tissue. In some embodiments, the anti-freeze proteins are configured to inhibit or limit the growth of ice-crystals, which can be sharp, needle-shaped ice crystals which form a fractional freeze pattern through the epidermal layer and/or dermal layer. Such anti-freeze proteins can be derived from one or more organisms including, for example, plants, lichens, fish, insects, bacteria, and/or crustaceans. Anti-freeze proteins may also be synthesized by recombinant expression in a suitable host organism (e.g., Escherichia coli) or by solid-phases peptide synthesis.

Anti-freeze proteins are grouped into one of four different types of anti-freeze proteins. These groups include type I, type II, type III, and anti-freeze glycoproteins, and are distinguished between groups by their molecular structures. For example, type I anti-freeze proteins refer to anti-freeze proteins with a primary structure comprised of a larger proportion of alanine amino acids residues relative to other amino acids and with a higher-order structure comprised primarily of α-helices.

As an example, type I anti-freeze proteins can be found in organisms such as flounders (i.e., Family: Paralichthys; Genus: Albigutta, Lethostigma, Dentatus, or Flesus, Family: Pseudopleuronectes; Genus: Americanus, Family: Hippog/ossus; Genus: Stenolepis,) sculpins (i.e., Family: Psychrolutidae; Genus: Psychrolutes, Neophrynichthys, Malacocottus, Gilbertidia, Eurymen, Ebinania, Dasycottus, Cottunculus, or Ambophthalmos), and/or Alaskan plaice (i.e., Family: Pleuronectes; Genus: Quadrituberculatus). Type II anti-freeze proteins refer to anti-freeze proteins with a primary structure that is comprised of a large proportion cysteine, alanine, asparagine, and glutanine amino acid residues relative to other amino acids and with a higher-order structure comprised primarily of β-sheets and random coils relative to α-helices.

As another example, type II anti-freeze proteins can be found in organisms such as American herring gull (i.e., Family: Larus; Genus: Smithsonianu), sea raven (i.e., Family: Hemitripteridae; Genus: Nautichthys, Hemitripterus, or Blepsais), poacher (i.e., Family: Agoninae; Genus: Sarritor, Podothecus, Leptagonus, Freemanichthys, Agonus, or Agonopsis), and/or smelt (i.e., Family: Osmeridae; Genus: Allosmerus, Hypomesus, Mallotus, Osmerus, Spirinchus, or Thaleichthys). Type III refers to anti-freeze proteins with a primary structure that is not primarily comprised of any particular amino acid residue and with a higher-order structure comprised primarily of β-sheets.

As another example, type III anti-freeze proteins can be found in organisms such as eel pout (i.e., Family: Zoarcidae; Subfamily: Gymnelinae, Lycodinae, Lycozoarcinae, or Zoarcinae), ocean pout (i.e., Family: Zoarces; Genus: Americanus, and wolfish (i.e., Family: Anarhichas; Genus: Lupus). Anti-freeze glycoproteins refer to anti-freeze proteins with a primary structure comprised of glycotripeptide repeats of alanine-alanine-threonine with a disaccharide galactose-N-acetylgalactosamine attached to each threonine and with higher-order structure that is not primarily comprised by α-helices relative to other protein folding types, such as β-sheets and random coils.

As yet another example, anti-freeze glycoproteins can be found in organisms such as Antarctic notothenioid (i.e., Family: Bovichtidea, Pseudaphritidae, Eleginopsidae, Nototheniidae, Harpagiferidae, Artedidraconidae, Bathydraconidae, or Channichthyidase) and/or northern cods (i.e., Family: Gadus; Genus: Morhua).

In several embodiments, the anti-freeze proteins of the freeze depressants provide a controlled zone of controlled ice crystal formation of certain portions of the epidermal, dermal layer and fat tissue, which in turn provides a discrete and targeted injury of cells forming the epidermal, dermal layer and fat tissue. Controlled formation of ice crystals in discrete zones occurs as anti-freeze proteins influence morphology of ice crystals as they form by freezing intracellular and/or extracellular water of the subject's epidermal, dermal layer and/or fat tissue. Anti-freeze proteins bind to the exterior of the ice crystals. In this way, anti-freeze proteins control ice-crystal morphology by directionally limiting growth of ice crystals in the epidermal, dermal layer and fat tissue as they reduce available surface area by which surrounding water molecules may bind. As such, this process slows and/or prevents the growth of larger crystals that would otherwise continue to propagate and ultimately result in uncontrolled damage to unfrozen cells and/or tissues

In these embodiments, the anti-freeze proteins create ice crystals with a needle-shape morphology by binding to the crystals so as to promote unidirectional crystal growth. Once an anti-freeze protein has bound to a growing ice crystal, growth propagates along one axis of the crystal unit cell (e.g., the c-axis). The needle-shaped crystals are then typified by a ratio in which the a-axis and b-axis of the crystal structure are approximately equal to one another whereas the c-axis is greater than either the a-axis or b-axis (e.g., a≈b and c>a or b). This morphology contrasts propagation of traditional crystal growth, which is multidirectional (e.g., indiscriminate growth in the a, b, and c axes), resulting in not only larger crystals, but also decreased uniformity. In some embodiments, anti-freeze proteins can also provide needle-shaped crystals that pack along their c-axes, resulting in the formation of vertically aligned needle-shaped crystals which creates a fractional freeze pattern through the epidermal layer and/or dermal layer. Without intending to be bound by any particular theory, vertically aligned, needle-shape crystals are thought to create less damage to the epidermal layer because they are smaller than traditional crystals, and because frozen epidermal columns are separated by unfrozen epidermal columns which promote tissue healing and minimize any adverse side effects.

In some embodiments, freeze depressant compositions having one or more anti-freeze proteins can create needle-shaped ice crystals of about 10 μm in length or less, such as needle-shaped ice crystals having a c-axis about 1 μm in length or greater and about 10 μm in length or less. For example, the c-axis of the needle-shaped crystals can be at least about 1 μm in length or greater and about 2 μm in length or less, about 3 μm in length or less, about 4 μm in length or less, or about 5 μm in length or less. In some embodiments, the needle-shaped ice crystals created by freeze depressant compositions having one or more anti-freeze proteins are about 1000 times smaller than traditional ice crystals, about 500 times, about 100 times, about 10 times, or about 5 times smaller than traditional ice crystals.

In some compositions, addition or an increase in concentration of solutes in the freeze depressant can be used to form a hypertonic formulation that locally dehydrates non-lipid-rich tissue (e.g., via osmotic dehydration). For example, the composition can include sodium salts (e.g., sodium chloride), calcium salts (e.g., calcium chloride), potassium salts (e.g., potassium chloride, potassium acetate), magnesium salts (e.g., magnesium chloride), ammonium sulphate and related compounds.

In further embodiments, the freeze depressant compositions can include hydrophilic and/or lipophobic molecules that favorably partition the freeze depressant within the upper layers (e.g., the epidermis and dermis) of the skin. Examples of hydrophilic molecules can include many compounds, especially those that reduce the surface tension of water, such as surfactants, gelatins, and hydrogels. In one embodiment, the freeze depressant includes glycolic acid that is completely miscible in water and is hydrophilic. Examples of lipophobic molecules can include fluorocarbons, which are typically non-polar and immiscible in water.

The freeze depressant may also include other additives in addition to or in lieu of the composition components described above. For example, some of the embodiments of freeze depressant compositions may also include a coloring agent, fragrance or perfume, emulsifier, stabilizer, an anesthetic agent, and/or other ingredient.

In a particular embodiment, the freeze depressant may include about 30% propylene glycol, about 30% glycerin, and about 40% ethanol by weight. In another embodiment, the freeze depressant may include about 40% propylene glycol, about 0.8% hydroxyethyl cellulose, and about 59.2% water by weight. In a further embodiment, the freeze depressant may include about 50% polypropylene glycol, about 40% glycerin, and about 10% ethanol by weight. In yet another embodiment, the freeze depressant may include about 59.5% water, about 40% propylene, and about 0.5% hydroxyethyl cellulose by weight.

In other embodiments, the freeze depressant includes about 10% of one or more type I anti-freeze proteins, about 20% of one or more type II anti-freeze proteins, about 30% of one or more type III anti-freeze proteins, and about 40% of one or more anti-freeze glycoproteins. In another embodiment, the freeze depressant includes about 20% of one or more type I anti-freeze proteins, about 10% of one or more type II anti-freeze proteins, about 30% of one or more type III anti-freeze proteins, and about 40% of one or more anti-freeze glycoproteins. In another embodiment, the freeze depressant includes about 10% of one or more type I anti-freeze proteins, about 30% of one or more type II anti-freeze proteins, about 20% of one or more type III anti-freeze proteins, and about 40% of one or more anti-freeze glycoproteins. In another embodiment, the freeze depressant includes about 40% of one or more type I anti-freeze proteins, about 30% of one or more type II anti-freeze proteins, about 20% of one or more type III anti-freeze proteins, and about 10% of one or more anti-freeze glycoproteins. In another embodiment, the freeze depressant includes about 50% of one or more type I anti-freeze proteins, about 10% of one or more type II anti-freeze proteins, about 20% of one or more type III anti-freeze proteins, and about 20% of one or more anti-freeze glycoproteins.

In other embodiments, the freeze depressant can include about 30-40% propylene glycol or polypropylene glycol. In one embodiment, the freeze depressant can include about 30-50% by volume of one or more freezing point depressants. Some freeze depressant compositions can further include 50% wt./vol. to about 70% wt./vol. of a combination of one or more of a thickening agent, a pH buffer, a humectant, a surfactant, and one more additives that (a) facilitate permeation of the freeze depressant into the epidermis and dermis, (b) increase an intracellular concentration of solutes of dermal and epidermal cells, and/r (c) form a hypertonic freeze depressant formulation and/or may include hydrophilic and/or lipophobic molecules. Some freeze depressant compositions can further include 0.005% wt./vol to 50% wt./vol of one or more anti-freeze proteins.

In other embodiments, the freeze depressant may include a concentration of greater than or equal to about 1 ng/mL to less than or equal to about 100 mg/mL of an anti-freeze protein. In another embodiment, the freeze depressant may include a concentration of 1 ng/mL to 10 ng/mL, 1 ng/mL to 100 ng/mL, 1 ng/mL to 10 μg/mL, 1 ng/mL to 100 μg/mL, or 1 ng/mL to 1 mg/mL of an anti-freeze protein. In another embodiment, the freeze depressant may include a concentration of 1 μg/mL to 10 mg/mL, 100 μg/mL to 10 mg/mL, or 10 μg/mL to 10 mg/mL of an anti-freeze protein.

In further embodiments, the freeze depressant composition can include one or more freezing point depressants in an amount between about 25% wt./vol. and about 55% wt. vol., about 30% wt./vol. and about 50% wt./vol., about 30% wt./vol. and about 40% wt./vol., about 35% wt./vol. and about 48% wt./vol. about 35% wt./vol. and about 45% wt./vol., about 38% wt./vol. and about 42% wt./vol. about 40% wt./vol. and about 50% wt./vol., about 40% wt./vol. and about 45% wt./vol.; or, in other embodiments, greater than about 30% wt./vol., about 35% wt./vol., about 40% wt./vol., about 45% wt./vol., or about 50% wt./vol.

In other embodiments, the freeze depressant composition can include a combination of one or more of a thickening agent, a pH buffer, a humectant, a surfactant, and an anti-freeze protein that (a) facilitates the formation of a fractional freeze pattern comprised of vertically aligned needle-shaped crystals through the dermal and/or epidermal layer, (b) provides controlled and discrete damage to targeted cells of the dermal layer and/or fat tissue through microzone freeze damage (c) triggers a healing and/or inflammatory response and (d) results in even and tightened skin.

One embodiment of the present technology described above is that an operator may use lower treatment temperatures for selectively affecting lipid-rich cells of the subject without causing undue freezing damage to the non-lipid-rich cells in the epidermis and/or dermis of the subject. The applied freeze depressant may lower the freezing point of the skin of the subject or body fluid in the target region to at least reduce the risk of unwanted intracellular and/or extracellular ice formation at such low treatment temperatures.

Still another embodiment, associated with several of the embodiments described above, is that the additives, adjuvants, solutes, etc. in the freeze depressant can provide a variety of desired additional properties to the freeze depressant material, with minimal or no effect on the chemistry and rheological properties of the freeze depressant. Accordingly, the additives will not interfere with the ability of the freeze depressant to protect a subject's biological tissues from freezing. Further, various additives described herein will enhance and/or facilitate the ability of the freeze depressant to protect a subject's biological tissues from freezing or other types of damage.

As described herein, the freeze depressant can be used with the treatment system 100 to transdermally cool and selectively affect the patient's subcutaneous lipid-rich tissue while protecting non-lipid-rich cells (e.g., residing in epidermal and/or dermal layers) from being substantially affected at the reduced temperatures. Subcutaneous lipid-rich tissue can be treated for a variety of therapeutic and cosmetic body-contouring applications, such as reduction of adipose tissue residing in identified portions of the patient's body, such as chin, cheeks, arms, pectoral areas, thighs, calves, buttocks, abdomen, “love handles”, back, breast, etc. For example, use of the freeze depressant with the treatment system 100 to transdermally cool adipose tissue in the breast can be used for breast contouring and size reduction in a manner that facilitates protection of non-target tissue in the breast. Further examples include use of the freeze depressant and treatment system 100 to contour and/or reduce a volumetric size of love handles, abdominal fat, back fat, etc., without substantially affecting non-targeted cells (e.g., cells in the epidermal and/or dermal layers).

In another embodiment, the freeze depressant can be used with the treatment system 100 to cool the skin of the patient to selectively affect (e.g., injure, damage, kill) secreting exocrine glandular cells. For example, secreting glandular cells residing in axilla apocrine sweat glands can be targeted by the treatment system 100 for the treatment of hyperhidrosis. In another example, lipid-producing cells residing in or at least proximate to sebaceous glands (e.g., glandular epithelial cells) present in the dermis of a target region can be targeted by the treatment system 100 for the treatment of acne or other skin condition. The lipid-producing cells residing in and/or proximate to sebaceous glands contribute to production of sebum, a waxy and oily secretion that can contribute to acne. For example, the treatment system 100 can be configured to reduce a temperature of a dermal layer of skin to reduce the temperature of lipid-producing cells residing in or at least proximate to sebaceous glands such that the targeted lipid-producing cells excrete a lower amount of sebum, such that there are fewer lipid-producing cells resulting in less sebum production within the targeted sebaceous glands, or in another embodiment, such that the sebaceous glands are destroyed. The treatment system 100 can be configured, for example, to reduce a subject's acne by cooling acne-prone regions of the body, such as the face, back, shoulders and chest.

### G. Additional Methods and Compositions

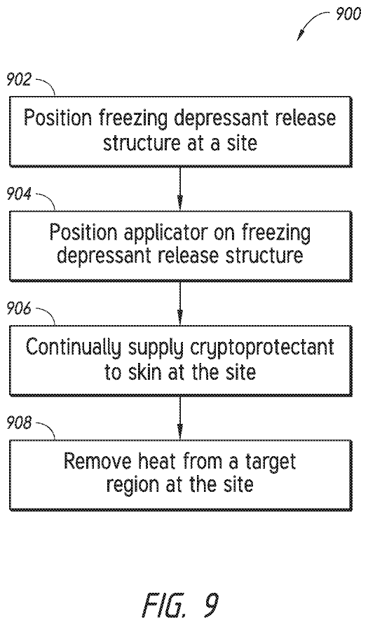

FIG. 9 is a flow chart illustrating a method 900 for cooling a site in accordance with embodiments of the invention. In some embodiments, heat can be applied to the site prior to introduction of cooling treatment for the destruction or alteration of lipid-rich cells. Even though the method 900 is described below with reference to the treatment system 100 of FIG. 1 and applicators 103, the method 900 may also be applied in other treatment systems with additional or different hardware and/or software components.

As shown in FIG. 9, the method 900 can positioning a freeze point depressant release structure at a site (block 902) and positioning an applicator on the freeze point depressant release structure (block 904). For example, the release structure having freeze point depressant absorbed therein can be placed on the skin of the subject at the site. In one embodiment, the freezing point depressant release structure can be positioned between a subject's skin and the applicator to facilitate sustained and/or replenishing release of the freeze point depressant to the skin during a treatment session. In another embodiment, the release structure is affixed to the applicator prior to positioning the release structure on the subject's skin. For example, surfaces of the applicator unit(s) can couple with the surface of the subject's skin at a target region. In one embodiment, the applicator unit can include a heat-exchanging unit, a heat-exchanging plate or cooling plate. In another embodiment, the surface of the applicator unit can be the surface of an interface layer or a patient protection sleeve/liner. Coupling of the surface(s) of the applicator unit(s) to the surface of the skin can be facilitated by using restraining means, such as a belt or strap. In other embodiments, a force (e.g., vacuum or suction force) can be used to positively couple the subject's skin at the target region to the surfaces.

The method 900 can continue by continually supplying a freeze point depressant to the subject's skin at the site (block 906). In some embodiments, the method can include continually supplying freeze point depressant to the skin of the subject which may maintain a sufficient concentration of absorbed freeze point depressant in the epidermis and/or dermis of the subject at the site for reducing the risk of freezing damage. The freeze point depressant composition supplied during a treatment session can be the same composition or, in other embodiments, a different composition than the freeze point depressant composition initially applied in step 906. Treatment duration can vary based on the purpose of the treatment, such as affecting one or more structures in the dermis (e.g., sebaceous glands, sweat glands, collagen and elastin fibers, and hair follicles), tightening skin, and/or cryolipolysis. For example, treatment can be applied for about 30 minutes, about 20 minutes, about 15 minutes, about 10 minutes, about 5 minutes, about 1 minute, or about 30 seconds to affect one or more structures in the dermis whereas treatment can be applied for about 3 hours, about 2.5 hours, about 2 hours, about 1.5 hours, about 1 hour, about 45 minutes, or about 30 minutes to tighten the subject's skin and/or cause cryolipolysis.

The method 900 can also include removing heat from the site of the subject (e.g., human or animal patient) during a treatment process selectively to cool cells in the site to a temperature below normal body temperature (block 908). For example, the lipid-rich tissue can be cooled to a temperature below about 37° C., below about 20° C., below about 10° C. or below about 0° C. such that lipid-rich cells are affected without substantially affecting non-lipid-rich cells. In some embodiments, the lipid-rich tissue can be cooled to about −20° C. to about 20° C., to about −18° C. to about 5° C. or to about −15° C. to about 0° C.

In further embodiments, methods that facilitate uptake (e.g., absorption) of freeze point depressant in the dermal and epidermal skin layers (e.g., across the stratum corneum) prior to or during cooling treatment can also include applying mechanical stimulation/agitation of the skin at the treatment site prior to introduction of cooling treatment for the destruction or alteration of lipid-rich cells.

Various embodiments of the methods described herein (e.g., method 900) can include a cosmetic treatment method for treating the target region of a human subject's body to achieve a cosmetically beneficial alteration of subcutaneous adipose tissue, a reduction in undesirable sweat secretion, or reduction in sebum secretion. Such a method could be administered by a non-medically trained person.

One expected advantage of several of the embodiments of the methods described herein (e.g., method 900) is that an operator may use lower treatment temperatures for selectively affecting lipid-rich cells of the subject without causing freezing damage to the dermal and epidermal tissue layers of the subject. The applied freezing point depressant compositions (e.g., freeze depressant) may lower the freezing point of the skin of the subject or body fluid in the target region to at least reduce the risk of intracellular and/or extracellular ice formation at such low treatment temperatures. Additionally, embodiments of the methods described herein (e.g., method 900) enhance loading and/or retention of the freeze depressant in the epidermal and dermal layers.

Another expected advantage of some of the embodiments of described herein (e.g., method 900) is that the dermis and/or epidermis of the subject may be continually protected against freezing damage due to the sustaining and/or replenishing administration of freeze depressant, and/or due to the administration of freeze depressant formulations disclosed herein.

In other embodiments, pre-treatment and/or post-treatment compositions can be provided to increase actual or a subject's perception of efficacy associated with a cooling treatment for aesthetic benefit. For example, a pre-treatment or post-treatment composition can include an anesthetic (e.g., benzocaine, lidocaine, butamben, pramoxine, tetracaine), cosmeceuticals (e.g., Daucus carota sativa extract, perfluorodecalin, perfluoro-n-octane), skin conditioners (e.g., squalene, dimethicone, divinyldimethicone, silsesquioxane crosspolymer and related compounds), anti-aging pro-collagen elements (e.g., glycolic acid, superoxide dismutase, niacinamide), fragrances, etc. In other embodiments, a pre-treatment or post-treatment composition can include menthyl lactate or related compounds that may enhance or promote vasoconstriction and/or impart a cooling sensation.

In various embodiments, pre-treatment and/or post-treatment compositions can be used in combination with other embodiments of the technology described herein. For example, a composition can be administered either during or before performing methods of at least some embodiments of the present invention, such as method 900. Likewise, post-treatment compositions can be administered at the conclusion of any treatment for removing heat from a treatment site to selectively affect lipid-rich cells.

In many embodiments, a series of substances can be applied to the target region during the course of a treatment. For example, a first substance can be a pre-treatment composition, second and third substances can include a first freeze depressant applied prior to heat removal from the site and a second freeze depressant applied to the site (e.g., in conjunction with the applicator) during the heat removal/cooling portion of the treatment. A fourth substance can be applied to the site the cooling process. Such a substance can be a post-treatment formulation. In particular, various treatments can include the application of one or more substances applied in series and/or, in other embodiments, simultaneously, to facilitate protection of non-targeted tissue and/or for tissue recovery post-treatment. Each substance can be adapted to (1) enhance the delivery or effect of a subsequently applied substance, (2) enhance the effect of cryotherapy, (3) reduce treatment times, and/or (4) reduce adverse effects of cryotherapy. In certain embodiments, the substances may include compositions having the same or at least similar formulations. For example, the application of a second substance may be simply the re-application or replenishment of the first substance. In other embodiments, the substances applied in series may comprise different compositions. In such embodiments, the earlier applied substances may be wiped or cleaned from the surface of the skin at the site prior to application of the next substance to be applied in series. In other embodiments, the later-applied substance(s) can be added to remaining earlier-applied substances at the surface of the skin.

The system 100 (FIG. 1) can be used to perform several pre-treatment and treatment methods. Although specific examples of methods are described herein, one skilled in the art is capable of identifying other methods that the system could perform. Moreover, the methods described herein can be altered in various ways. As examples, the order of illustrated logic may be rearranged, sub-stages may be performed in parallel, illustrated logic may be omitted, other logic may be included, etc.

### H. Suitable Computing Environments

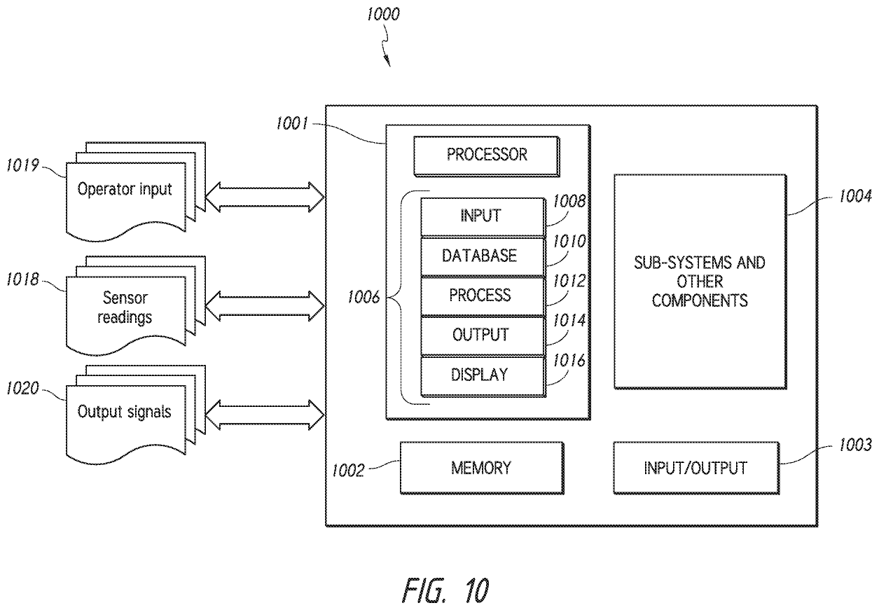

FIG. 10 is a schematic block diagram illustrating subcomponents of a computing device 1000 in accordance with an embodiment of the disclosure. The computing device 1000 can include a processor 1001, a memory 1002 (e.g., SRAM, DRAM, flash, or other memory devices), input/output devices 1003, and/or subsystems and other components 1004. The computing device 1000 can perform any of a wide variety of computing processing, storage, sensing, imaging, and/or other functions. Components of the computing device 1000 may be housed in a single unit or distributed over multiple, interconnected units (e.g., though a communications network). The components of the computing device 1000 can accordingly include local and/or remote memory storage devices and any of a wide variety of computer-readable media.

As illustrated in FIG. 10, the processor 1001 can include a plurality of functional modules 1006, such as software modules, for execution by the processor 1001. The various implementations of source code (i.e., in a conventional programming language) can be stored on a computer-readable storage medium or can be embodied on a transmission medium in a carrier wave. The modules 1006 of the processor can include an input module 1008, a database module 1010, a process module 1012, an output module 1014, and, optionally, a display module 1016.

In operation, the input module 1008 accepts an operator input 1019 via the one or more input devices described above with respect to FIG. 1, and communicates the accepted information or selections to other components for further processing. The database module 1010 organizes records, including patient records, treatment data sets, treatment profiles and operating records and other operator activities, and facilitates storing and retrieving of these records to and from a data storage device (e.g., internal memory 1002, an external database, etc.). Any type of database organization can be utilized, including a flat file system, hierarchical database, relational database, distributed database, etc.

In the illustrated example, the process module 1012 can generate control variables based on sensor readings 1018 from sensors (and/or other data sources), and the output module 1014 can communicate operator input to external computing devices and control variables to the controller 114 (FIG. 1). The display module 1016 can be configured to convert and transmit processing parameters, sensor readings 1018, output signals 1020, input data, treatment profiles, and prescribed operational parameters through one or more connected display devices, such as a display screen, printer, speaker system, etc. A suitable display module 1016 may include a video driver that enables the controller 114 to display the sensor readings 1019 or other status of treatment progression on the output device 120 (FIG. 1).

In various embodiments, the processor 1001 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 1002 can be standard memory, secure memory, or a combination of both memory types. By employing a secure processor and/or secure memory, the system can ensure that data and instructions are both highly secure and sensitive operations such as decryption are shielded from observation.

Suitable computing environments and other computing devices and user interfaces are described in commonly assigned U.S. Pat. No. 9,275,442, entitled “TREATMENT PLANNING SYSTEMS AND METHODS FOR BODY CONTOURING APPLICATIONS,” which is incorporated herein in its entirety by reference.

### I. Conclusion

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or blocks are presented in a given order, alternative embodiments may perform routines having stages, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times. The headings provided herein are for convenience only and do not interpret the scope or meaning of the described technology.

The terminology used in the description is intended to be interpreted in its broadest reasonable manner, even though it is being used in conjunction with a detailed description of identified embodiments.

The term “about” as used herein means within 10% of a stated value or range of values.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense of one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense of one having skill in the art would understand the convention (e.g., “a system having at least one of A, B, or C” would include but not be limited to systems that have A alone, B alone, C alone, A and B together, A and C together, B and C together, and/or A, B, and C together, etc.).

Some of the functional units described herein have been labeled as modules, in order to more particularly emphasize their implementation independence. For example, modules (e.g., modules discussed in connection with FIG. 9) may be implemented in software for execution by various types of processors. An identified module of executable code may, for instance, comprise one or more physical or logical blocks of computer instructions which may, for instance, be organized as an object, procedure, or function. The identified blocks of computer instructions need not be physically located together, but may comprise disparate instructions stored in different locations which, when joined logically together, comprise the module and achieve the stated purpose for the module.

A module may also be implemented as a hardware circuit comprising custom VLSI circuits or gate arrays, off-the-shelf semiconductors, such as logic chips, transistors, or other discrete components. A module may also be implemented in programmable hardware devices, such as field programmable gate arrays, programmable array logic, programmable logic devices or the like.

A module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

Any patents, applications and other references cited herein, are incorporated herein by reference. Embodiments of the described technology can be modified, if necessary, to employ the systems, functions, and concepts of the various references described above to provide yet further embodiments.

These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or embodiments of the technology should not be taken to imply that the terminology is being redefined herein to be restricted to any specific characteristics, features, or embodiments of the technology with which that terminology is associated.

I/We claim:

1. A method for affecting a subcutaneous layer of a human subject's body, the method comprising:
applying a freezing point depressant to a surface of the human subject's skin at a site; and
freezing extracellular and/or intracellular water of a dermal layer and/or fat tissue in the human subject to form a fractional freeze pattern comprised of needle-shaped ice crystals through an epidermal and dermal layer by puncturing a cell membrane of a frozen cell within the site with the needle-shaped ice crystals to cause cell death.

2. The method of claim 1, wherein the freezing occurs in a targeted microzone.

3. The method of claim 1, wherein the cell death initiates an increased production of collagen and/or elastin.

4. The method of claim 1, wherein the subject exhibits minimal damage to the epidermal layer during freezing of the extracellular and/or intracellular water of a dermal layer and/or the fat tissue.

5. The method of claim 1, further comprising triggering a healing and/or inflammatory response in the human subject's body.

6. The method of claim 1, wherein the freezing point depressant further includes at least one of a thickening agent, a pH buffer, a humectant, or a surfactant.

7. A method for affecting a target region of a human subject's body, the method comprising:
positioning a freeze depressant release structure having an array of openings extending into a body of the freeze depressant release structure at a region on a surface of the human subject's skin;
delivering a freezing point depressant having one or more anti-freeze proteins from a carrier material forming the freeze depressant release structure to a surface of skin at the region; and
removing heat from the target region of the human subject to cool subcutaneous lipid-rich cells in the region to a temperature below normal body temperature.

8. The method of claim 7, wherein removing heat from the target region of the human subject further comprises removing heat from one or more portions of the target region to cool one or more structures and/or one or more cells in the target region.

9. The method of claim 8, wherein the one or more portions of the target region include the subject's dermal and/or hypodermal layers of the subject's skin.

10. The method of claim 9, wherein the one or more portions of the target region include the subject's epidermal layer of the subject's skin.

11. The method of claim 10, wherein removing heat from the target region of the human subject further comprises creating one or more discrete partially frozen microchannels in the epidermal layer of the subject's skin which are separated by unfrozen areas of the epidermal layer.

12. The method of claim 11, wherein removing heat from the region of the human subject generates a fractional freeze.

13. The method of claim 7, wherein the freezing point depressant is delivered to the region for about five minutes or less.

14. The method of claim 7, wherein, during delivery of the freezing point depressant, a temperature of one or more target structures within one or more of human subject's dermal structures is reduced.

15. The method of claim 14, wherein the one or more structures includes one or more sebaceous glands.

16. The method of claim 7, wherein positioning the freeze depressant release structure at the region further comprises contacting the human subject's skin with a heat removal apparatus coupled to a portion of the release structure.

17. The method of claim 16, wherein contacting the human subject's skin with a heat removal apparatus coupled to a portion of the release structure further comprises removing heat from the human subject's epidermis without causing heat removal injury to the epidermis.

18. The method of claim 17, wherein a portion of the region is contacted by a greater amount of the freezing point depressant compared to another portion of the region.

19. The method of claim 18, wherein the portion of the region contacted by the greater amount of freezing point depressant freezes slower compared to the another portion of the region.

20. The method of claim 7, wherein the anti-freeze proteins cause any water molecules that freeze to predominately freeze with a linear needle-like structure as opposed to a snowflake-like structure.
